(19) United States
(12) Patent Application Publication
(10) Pub. No.: US 2008/0279829 A1
(43) Pub. Date: Nov. 13, 2008

(54) Phenyl-(4-Phenyl-Pyrimidin-2-Yl)-Amines For Enhancing Immunotolerance

(76) Inventors: Maximilian Woisetschlaeger, Wien (AT); Ehud Hauben, Cerbusco sul Naviglio (IT); Maria Grazia Roncarolo, Segrate MI (IT)

Correspondence Address: NOVARTIS INSTITUTES FOR BIOMEDICAL RESEARCH, INC., 400 TECHNOLOGY SQUARE, CAMBRIDGE, MA, 02139, US

(21) Appl. No.: 12/088,550

(22) PCT Filed: Sep. 27, 2006
(86) PCT No.: PCT/EP2006/009406

§ 371 Date: Mar. 28, 2008

(30) Foreign Application Priority Data
Sep. 29, 2005 (GB) 0519838.7
Oct. 6, 2005 (GB) 0520372.4

(51) Int. Cl. A61K 35/12; A61P 37/02

(52) U.S. Cl. 424/93.7

(57) ABSTRACT

The use of a phenyl-(4-phenyl-pyrimidin-2-yl)-amine for the preparation of a medicament for enhancing immunotolerance in a mammal; and uses, methods, processes and pharmaceutical combinations and compositions comprising such phenyl-(4-phenyl-pyrimidin-2-yl)-amine.

The present invention relates to the use of phenyl-(4-phenyl-pyrimidin-2-yl)-amines for enhancing immunotolerance, e.g. in cell therapy.

Dendritic cells (DC) which are derived from monocytes, are highly effective antigen presenting cells that are capable of priming and stimulating T cell responses to a wide variety of antigens. As such, DCs have an effector function of both, immunity and tolerance and DCs play a critical role in the immune response against tumors as well as numerous bacterial and other pathogens. For a detailed discussion of DCs as well as numerous other topics of interest see, e.g. Paul (1998) Fundamental Immunology, 4^{th} edition, Lippincott-Raven, Philadelphia.

We have surprisingly found that phenyl-(4-phenyl-pyrimidin-2-yl)-amines may be useful in cell therapy, which cell therapy may be useful to enhance immunotolerance in a mammal, e.g. compared with the immunotolerance status before such treatment, e.g. phenyl-(4-phenyl-pyrimidin-2-yl)-amines may be useful as immunosuppressant.

In several aspects the present invention provides
1.1 The use of a phenyl-(4-phenyl-pyrimidin-2-yl)-amine for the preparation of a medicament for enhancing immunotolerance in a mammal.
1.2. The use of a phenyl-(4-phenyl-pyrimidin-2-yl)-amine for the preparation of a medicament for inducing, e.g. active inducing, e.g. long term, immunotolerance in a mammal.
1.3 The use of a phenyl-(4-phenyl-pyrimidin-2-yl)-amine for the preparation of a medicament for use as an immunosuppressant.
1.4 The use of a phenyl-(4-phenyl-pyrimidin-2-yl)-amine for the preparation of a medicament for use in cell therapy.

According to the present invention it was found that monocytes and/or dendritic cells isolated from a mammal, when matured in the presence of a phenyl-(4-phenyl-pyrimidin-2-yl)-amine may be useful to enhance immunotolerance in a mammal, e.g. compared with the immunotolerance status before such treatment.

In another aspect the present invention provides
2.1 A method for enhancing the immunotolerance in a mammal, or
2.2 A method for inducing, e.g. active inducing, e.g. long term, immunotolerance in a mammal,
2.3 A cell therapy
   comprising the steps of
   a) isolating monocytes and/or dendritic cells from a mammal,
   b) differentiating the monocytes, if present, into immature dendritic cells (iDC), optionally in the presence of an phenyl-(4-phenyl-pyrimidin-2-yl) amine,
   c) treating immature dendritic cells and dendritic cells obtained in step a) and/or b) with a maturation agent in the presence of an phenyl-(4-phenyl-pyrimidin-2-yl) amine, and
   d) administering an effective amount of maturated dendritic cells obtained in step c) to said mammal.

Methods of steps a) to d) according to the present invention may be carried out as conventional, e.g. according, such as analogously to a method as conventional.

E.g. methods to isolate functional monocytes and/or dendritic cells (DCs) from a mammal, e.g. from a blood sample of such mammal, are known and may be carried out according, e.g. analogously, to a method as conventional. E.g. isolating monocytes and/or dendritic cells (DCs) may be carried out by use of magnetic beads, such as present in commercially available kits, such as from Miltenyi Biotech according to the corresponding protocol, or by any other cell sorting method, e.g. according, e.g. analogously, to a method as conventional.

Monocytes may be differentiated into immature dendritic cells (iDC) by treatment with IL-4 and GM-CSF for several days, e.g. 4 to 8 days, and differentiation optionally may be carried out according to the present invention in the presence of a phenyl-(4-phenyl-pyrimidin-2-yl) amine, which may be present in the cell/differentiation mixture in an amount of (ca.) 1 nM to (ca.) 30 nM. i DCs obtained in such differentiation step or iDCs or DCs isolated in step a), e.g. from blood, are treated in step b) with a maturation agent in order to obtain mature DCs, e.g. in appropriate culture medium. An appropriate maturation agent may be used, e.g. including such as conventional. The maturation agent may e.g. comprise as an active maturation agent GM-CSF, LPS, IL-1-beta, IL-4, agonistic CD40-antibodies TNF-alpha, TSLP (thymic stromal lymphopoietin), or a combination of one or more of such active maturation agents cited. By treatment of iDCs with a maturation agent in appropriate culture the iDCs differentiate into mature DCs, e.g. by treatment for some hours, e.g. (ca.) 4 to (ca.) 36 hours, such as (ca.) 6 to (ca.) 24 hours. Appropriate differentiation and maturation culture (conditions) to obtain mature DCs from monocytes or iDCs are known or may be found by a person skilled in the art.

Maturation of DCs (and treatment of DCs) is carried out in the presence of an phenyl-(4-phenyl-pyrimidin-2-yl) amine. Such phenyl-(4-phenyl-pyrimidin-2-yl) amine may be present in the cell/maturation mixture in an amount of (ca.) 1 nM to (ca.) 30 nM.

Treatment with maturation agent and phenyl-(4-phenyl-pyrimidin-2-yl) amine should be followed by a purification step of the DCs obtained, e.g. by washing with an appropriate washing solution, e.g. PBS, in order to remove the maturation agent and phenyl-(4-phenyl-pyrimidin-2-yl) amine from the DCs obtained.

DCs obtained in step c) are administered to the mammal from which the untreated monocytes and/or DCs are isolated, e.g. such DCs treated in step c) are administered to the mammal by intravenous administration, e.g. by an appropriate method using an appropriate device, e.g. injection, infusion, in the form of a pharmaceutical composition. For intravenous administration DCs obtained in step c) are purified, e.g. as described above, and the purified DCs are administered in aqueous medium which is appropriate for intravenous administration, e.g. aqueous medium may comprise physiologically acceptable adjuvants, e.g. to obtain an aqueous medium which is similar to a physiological medium. Aqueous medium for administering intravenously cells to a mammal is known or may be found by a skilled person. The concentration of the DCs treated in aqueous medium according to the present invention for administration may be similar to physiological conditions, but in general a higher concentration than under physiological conditions is recommended in order to improve the effect in vivo.

In another aspect the present invention provides
3.1 A pharmaceutical composition, e.g. for intravenous administration, comprising mature dendritic cells which are matured and treated outside of the mammalian body in the presence of an phenyl-(4-phenyl-pyrimidin-2-yl)-amine, beside a pharmaceutically acceptable excipient, e.g. carrier, diluent.

A pharmaceutical composition comprising DCs treated according to the present invention may comprise aqueous medium; e.g. and beside aqueous medium optionally further pharmaceutically acceptable excipient, e.g. excipient which is appropriate for administering cells to a mammal. Aqueous medium comprises an appropriate buffer medium, e.g. a buffer medium which is useful and pharmaceutically acceptable for intravenous administration. A pharmaceutical composition provided by the present invention may be prepared as appropriate, e.g. according, e.g. analogously, to a method as conventional, e.g. preparing a composition comprising mature dendritic cells which are matured and treated outside of the mammalian body in the presence of a phenyl-(4-phenyl-pyrimidin-2-yl)-amine and from which phenyl-(4-phenyl-pyrimidin-2-yl)-amine and maturation agent has been removed, e.g. by isolating the cells, e.g. by washing the cells, which composition is appropriate for intravenous administration, e.g. in the form of an emulsion or a suspension of the DCs, e.g. in aqueous medium, e.g., buffer medium.

To the isolated cells optionally further pharmaceutically acceptable excipient, e.g. carrier or diluent, may be added.

In another aspect the present invention provides
4.1 A process for the production of a pharmaceutical composition, e.g. for intravenous administration, comprising
   a) maturing dendritic cells outside of the mammalian body in the presence of an phenyl-(4-phenyl-pyrimidin-2-yl)-amine,
   b) isolating the cells obtained in step a), and
   c) optionally mixing with pharmaceutically acceptable excipient.

Pharmaceutically acceptable excipient comprises carrier and diluent, e.g. aqueous medium, including physiologically acceptable buffer medium.

It was found that treatment according to the present invention surprisingly induces immunotolerance in a mammal, e.g. such treatment enhances immunotolerance in a mammal, compared with its immunotolerance-status before treatment.

E.g., cell therapy with bone-marrow-derived mature dendritic cells treated in vitro with a phenyl-(4-phenyl-pyrimidin-2-yl)-amine, e.g. such as Compound A, or compounds B as defined herein, significantly prevents islet graft rejection in mice, e.g. a phenyl-(4-phenyl-pyrimidin-2-yl)-amine promotes the induction of long-term active tolerance, e.g. independently of any additional immunosuppressive regimen.

The use or a method or a pharmaceutical composition provided by the present invention is herein also designated as “use (or a method, or pharmaceutical composition) of (according to) the present invention”.

Appropriate phenyl-(4-phenyl-pyrimidin-2-yl)-amines which are useful according to the present invention, e.g. for use, for a method or in a pharmaceutical composition according to the present invention, includes phenyl-(4-phenyl-pyrimidin-2-yl)-amines such as disclosed in WO03063871. Phenyl-(4-phenyl-pyrimidin-2-yl)-amines which are useful according to the present invention are herein also designated as “phenyl-(4-phenyl-pyrimidin-2-yl) amine(s) of (according to) the present invention”.

In one aspect a phenyl-(4-phenyl-pyrimidin-2-yl) amine according to the present invention is a compound of formula

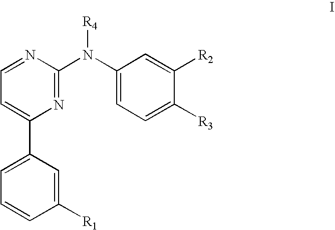

wherein
R₁ is halogen or halo(C₁₋₄)alkyl,
R₂ is hydrogen, halogen or halo(C₁₋₄)alkyl,
R₃ is halogen or halo(C₁₋₄)alkyl,
R₄ is hydrogen, (C₁₋₈)alkyl, hydroxy(C₁₋₆)alkyl or a group of formula
—CO—R₅,
—CO—(CH₂)ₘ—OR₆,
—CO—CO—R₇,
—CO—CO—OR₈,
—CO—N(R₉R₁₀),
—CO—(CH₂), —CO—R₁₁,
—CO—(CHR₁₅)—O—(CH₂)ₒ—CO—R₁₁,
—CO—(CH₂)ₚ—O—(CH₂)_{q}—O—(CH₂)ᵣ—R₁₆,
—CO—O—(CH₂)ₛ—O—CO—R₁₇,
—CO—O—(CH₂)ₜN(R₁₈R₁₉),
—CO—O—(CH₂)ᵤ—NH—CO—CH(NH₂)—R₂₀, or
—CO—O—(CH₂)_{w}—NH—CO—R₁₇,
   R₅ is hydrogen, (C₁₋₈)alkyl, (C₃₋₈)cycloalkyl, amino, (C₁₋₄)alkylamino, di(C₁₋₄)alkylamino, aryl, e.g. (C₆₋₁₈)aryl, or heterocyclyl, e.g. having 5 or 6 ring members and 1 to 4 heteroatoms selected from N, O or S,
   R₆ is hydrogen, (C₁₋₄)alkyl, (C₃₋₈)cycloalkyl, aryl, e.g. (C₆₋₁₈)aryl, (C₁₋₄)alkyl substituted by heterocyclyl having 5 or 6 ring members and 1 to 4 heteroatoms selected from N, O or S, amino(C₁₋₆)alkyl, (C₁₋₄)alkylamino(C₁₋₆)alkyl, di(C₁₋₄)alkylamino(C₁₋₆)alkyl, hydroxy(C₁₋₆)alkyl, hydroxy(C₁₋₄)alkylamino(C₁₋₆)alkyl or an amino acid residue, e.g. an amino acid residue of formula —CH₂—CH(NH₂)—COOH, or, e.g. an amino acid residue which amino acid residue is obtainable by reacting a compound of formula I wherein R₆ is —CO—(CH₂)ᵥ—CO—Cl with an amino acid, an amino acid mono(C₁₋₆)alkyl ester or an amino acid di(C₁₋₄)alkyl ester,
   R₇ and R₈ independently of each other are (C₁₋₄)alkyl, (C₃₋₈)cycloalkyl, aryl or heterocyclyl having 5 or 6 ring members and 1 to 4 heteroatoms selected from N, O or S,
   R₉ and R₁₀ independently of each other are hydrogen or (C₁₋₄)alkyl or
   one of R₉ and R₁₀ is hydrogen and the other is (C₃₋₈)cycloalkyl, (C₁₋₄)alkyl, aryl, e.g. (C₆₋₁₈)aryl, or heterocyclyl having 5 or 6 ring members and 1 to 4 heteroatoms selected from N, O or S,
   R₁₁ is (C₁₋₄)alkyl, —OR₁₂, —NR₁₃R₁₄, an amino acid residue, e.g. which amino acid residue is obtainable by reacting a compound of formula I wherein R₁₁ is —CO—(CHR₁₅)—O—(CH₂)ₒ—CO—Cl with an amino acid, an amino acid mono(C₁₋₆)alkyl ester or an amino acid di(C₁₋₆)alkyl ester,
   R₁₂ is hydrogen or (C₁₋₄)alkyl, such as an amino acid residue, wherein the binding is effected via its amine group;
   R₁₃ and R₁₄ independently of each other are hydrogen, (C₁₋₄)alkyl, amino(C₁₋₆)alkyl, (C₁₋₄)alkylamino(C₁₋₆)alkyl, di(C₁₋₄)alkylamino(C₁₋₆)alkyl,
   R₁₅ is hydrogen or (C₁₋₄)alkyl,
   R₁₆ is hydrogen, (C₁₋₄)alkyl, carboxyl or a carboxylic ester residue, e.g. attached via its carbonyl group;
   R₁₇ is amino(C₁₋₄)alkyl, (C₁₋₄)alkylamino(C₁₋₄)alkyl or di(C₁₋₄)alkylamino(C₁₋₄)alkyl,
   R₁₈ is hydrogen or (C₁₋₄)alkyl,
   R₁₉ is hydroxy(C₁₋₄)alkyl,
   R₂₀ is (C₁₋₄)alkyl or hydroxy(C₁₋₄)alkyl,
   m is 0 to 4, n is 2 to 8, o is 0 to 4, p is 0 to 4, q is 1 to 8, r is 0 to 4, s is 1 to 4, t is 1 to 4, u is 1 to 6, and w is 1 to 6.

In another aspect a phenyl-(4-phenyl-pyrimidin-2-yl) amine according to the present invention is a compound of formula I, wherein

R₁ is chloro or trifluoromethyl,
R₂ is hydrogen or trifluoromethyl,
R₃ is chloro, fluoro or trifluoromethyl,
R₄ is hydrogen, (C₁₋₄)alkyl, e.g. methyl, hydroxy(C₁₋₄)alkyl, e.g. hydroxyethyl, or a group of formula
—CO—R₅,
—CO—(CH₂)ₘ, —OR₆,
—CO—CO—R₇,
—CO—CO—OR₈,
—CO—N(R₉R₁₀),
—CO—(CH₂), —CO—R₁₁,
—CO—(CHR₁₅)—O—(CH₂)ₒ—CO—R₁₁,
—CO—(CH₂)ₚ—O—(CH₂)_{q}—O—(CH₂)ᵣ—R₁₆,
—CO—O—(CH₂), —O—CO—R₁₇,
—CO—O—(CH₂)ₜ—N(R₁₈R₁₉),
—CO—O—(CH₂)ᵤ—NH—CO—CH(NH₂)—R₂₀, or
—CO—O—(CH₂)_{w}—NH—CO—R₁₇,
   R₅ is hydrogen, (C₁₋₄)alkyl, (C₃₋₆)cycloalkyl, dimethylamino, phenyl or heterocyclyl having 5 or 6 ring members and 1 to 4 heteroatoms selected from N, O or S, such as heterocyclyl having 6 ring members and one 0 as a heteroatom, e.g. tetrahydropyranyl,
   R₆ is hydrogen, (C₁₋₄)alkyl, (C₁₋₂)alkyl substituted by heterocyclyl having 5 or 6 ring members and 1 to 4 heteroatoms selected from N, O or S, such as heterocyclyl having 5 or 6 ring members and having 1 or 2 heteroatoms selected from N or O, e.g. including unsubstituted pyrrolidine, morpholine and piperazine and piperazine substituted by e.g. (C₁₋₂)alkyl or (C₁₋₂)hydroxyalkyl; amino(C₁₋₄)alkyl, (C₁₋₂)alkylamino(C₁₋₄)alkyl, di(C₁₋₂)alkylamino(C₁₋₄)alkyl, hydroxy(C₁₋₃)alkyl, hydroxy(C₁₋₂)alkylamino(C₁₋₂)alkyl or an amino acid residue, e.g. —CH₂—CH(NH₂)—COOH,
   R₇ and R₈ independently of each other are (C₁₋₂)alkyl or phenyl,
   R₉ and R₁₀ independently of each other are hydrogen or (C₁₋₂)alkyl,
   R₁₁, is (C₁₋₂)alkyl, —OR₁₂, —NR₁₃R₁₄, an amino acid residue, an (C₁₋₂)alkylester thereof or an di(C₁₋₂)alkylester thereof, preferably originating from an amino acid selected from the group consisting of alanine, phenylalanine, glutamic acid and lysine, wherein the binding is effected via the α-amino group or, e.g. in the case of lysine via the ε-amino group,
   R₁₂ is hydrogen or (C₁₋₂)alkyl,
   R₁₃ and R₁₄ independently of each other are hydrogen, (C₁₋₂)alkyl, amino(C₁₋₄)alkyl, (C₁₋₂)alkylamino(C₁₋₄)alkyl, di(C₁₋₂)alkylamino(C₁₋₄)alkyl,
   R₁₅ is hydrogen or (C₁₋₂)alkyl,
   R₁₆ is hydrogen, (C₁₋₂)alkyl, carboxyl or carboxylic ester, e.g. wherein attached via its carbonyl group;
   R₁₇ is amino(C₁₋₂)alkyl,
   R₁₈ is hydrogen or (C₁₋₂)alkyl,
   R₁₉ is hydroxy(C₁₋₂)alkyl,
   R₂₀ is (C₁₋₂)alkyl or hydroxy(C₁₋₂)alkyl,
   m is 0 or 1, n is 2 to 4, o is 0 or 1, p is 0 to 2, q is 2 to 5, r is 0 to 2, s is 2, t is 2,
   u is 1 to 3 and w is 1 to 3.

A compound of formula I, wherein R₄ is other than hydrogen is expected to be a precursor of a compound of formula I, wherein R₄ is hydrogen, e.g. a compound of formula I, wherein R₁ is chloro, R₂ is hydrogen, R₃ is trifluoromethyl and R₄ is a group of formula —CO—O—(CH₂)₂—N[(C₂H₅OH)(CH₃)] (herein also designated as “Compound A”) has been found to be a precursor of a compound of formula I, wherein R₁ is chloro, R₂ is hydrogen, R₃ is trifluoromethyl and R₄ is hydrogen (herein also designated as “Compound B”) which has been found to be the active compound.

In another aspect a phenyl-(4-phenyl-pyrimidin-2-yl) amine according to the present invention is a compound of formula I, which is selected from the group consisting of
N-[4-(3-Chloro-phenyl)-pyrimidin-2-yl]-N-(4-chloro-3-trifluoromethyl-phenyl)-amine,
N-[4-(3-Trifluoromethyl-phenyl)-pyrimidin-2-yl]-N-(4-fluoro-3-trifluoromethyl-phenyl)-amine,
N-[4-(3-Trifluoromethyl-phenyl)-pyrimidin-2-yl]-N-(4-chloro-3-trifluoromethyl-phenyl)-amine,
N-[4-(3-Trifluoromethyl-phenyl)-pyrimidin-2-yl]-N-(4-trifluoromethyl-phenyl)-amine, and
N-[4-(3-Chloro-phenyl)-pyrimidin-2-yl]-N-(4-trifluoromethyl-phenyl)-amine,
   wherein the amine group is unsubstituted,
   wherein the amine group is substituted by R₄, wherein R₄ is as defined above.

In another aspect a phenyl-(4-phenyl-pyrimidin-2-yl)-amine according to the present invention is selected from the group consisting of compounds of formulae

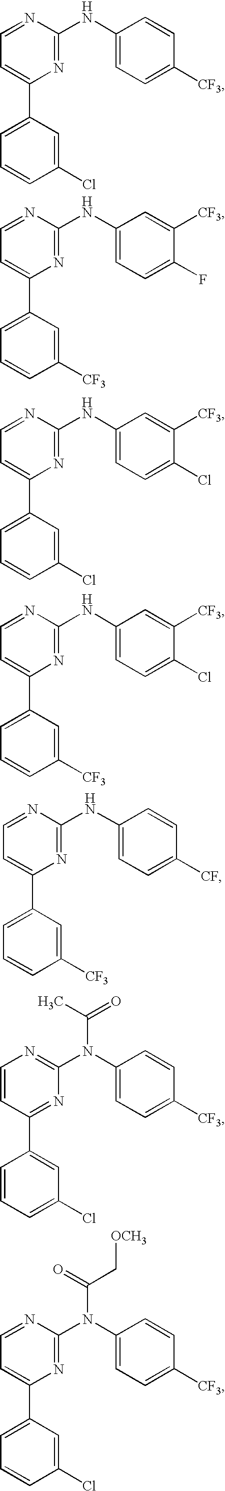

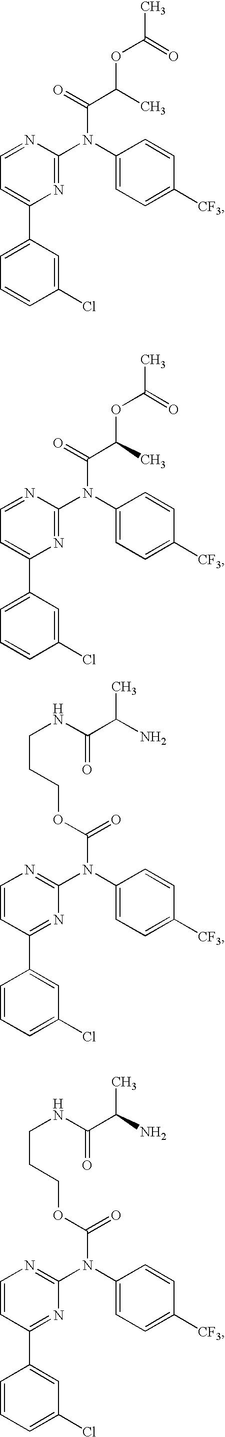

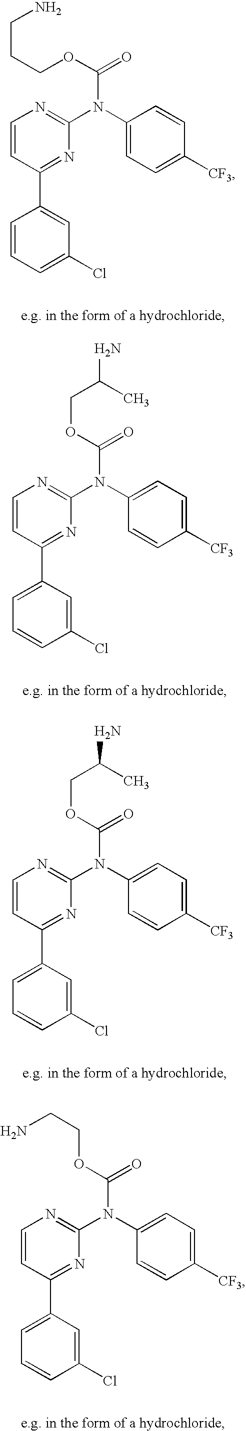

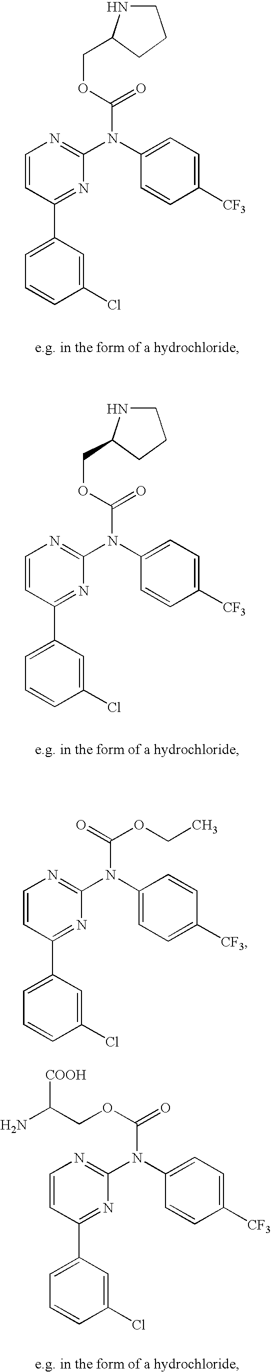

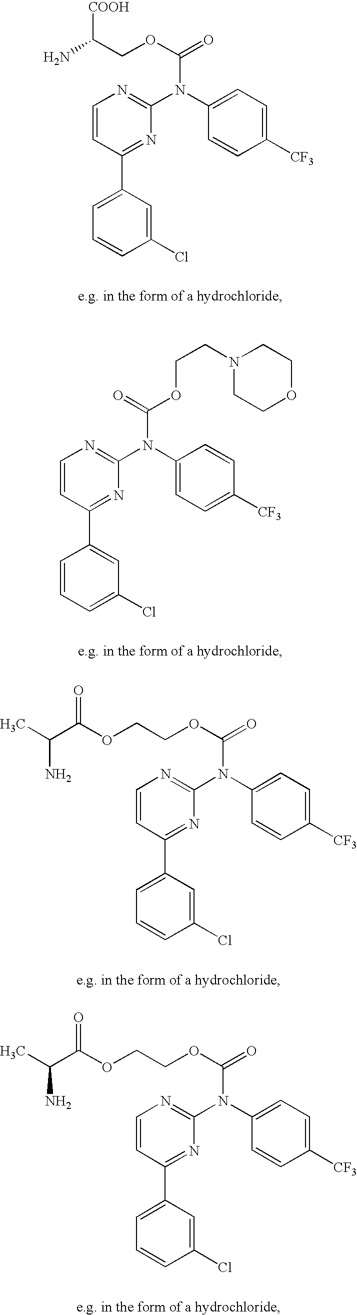

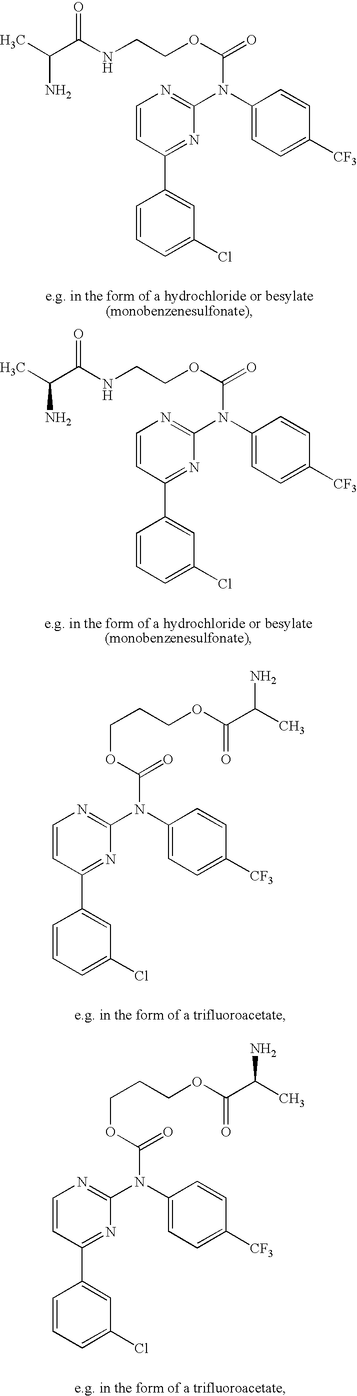

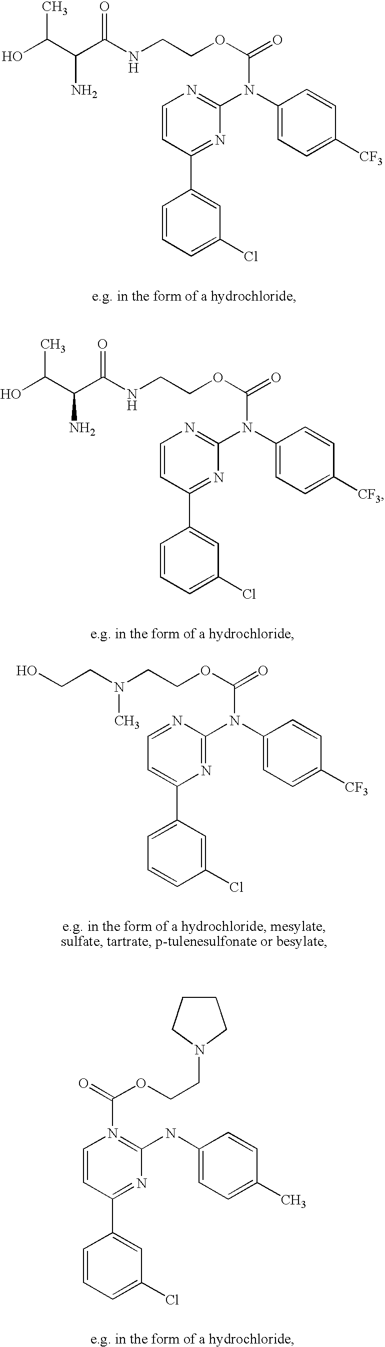

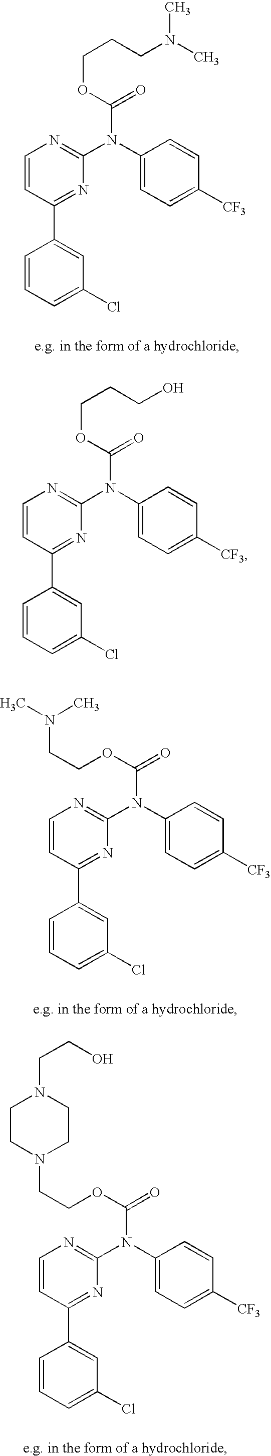

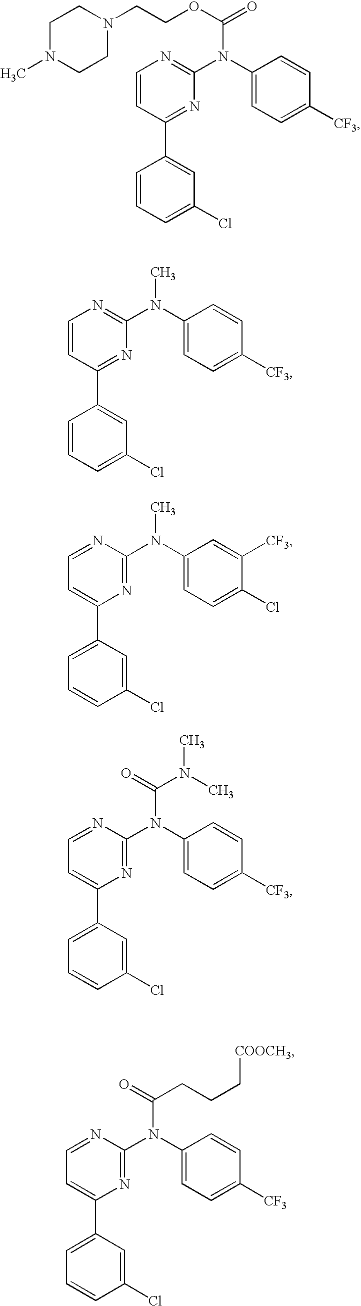

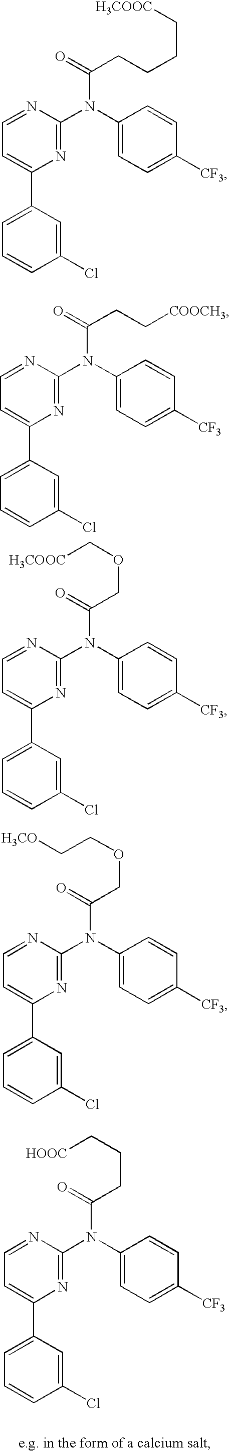

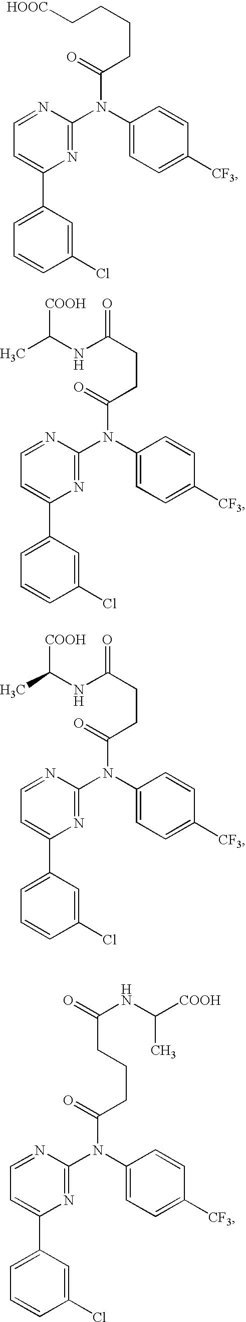

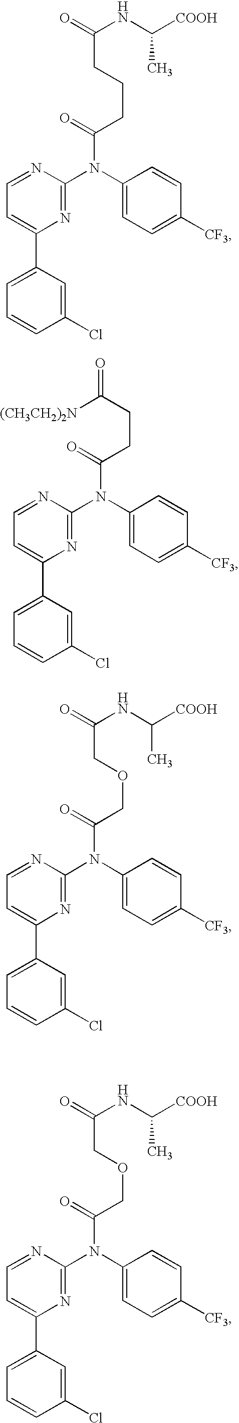

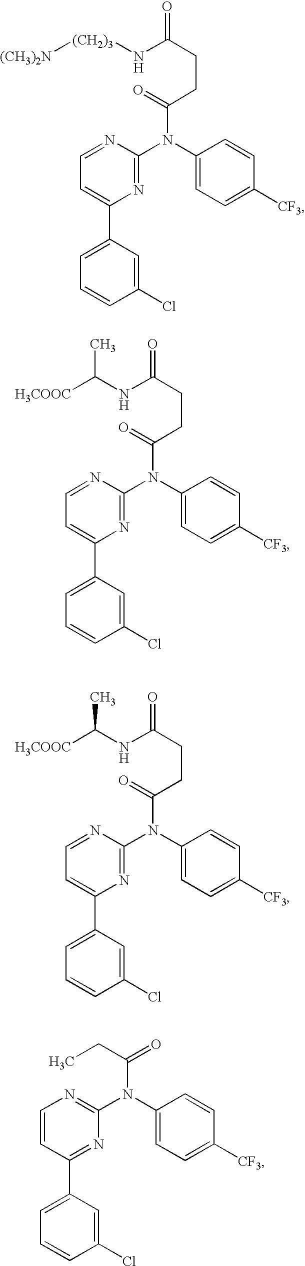

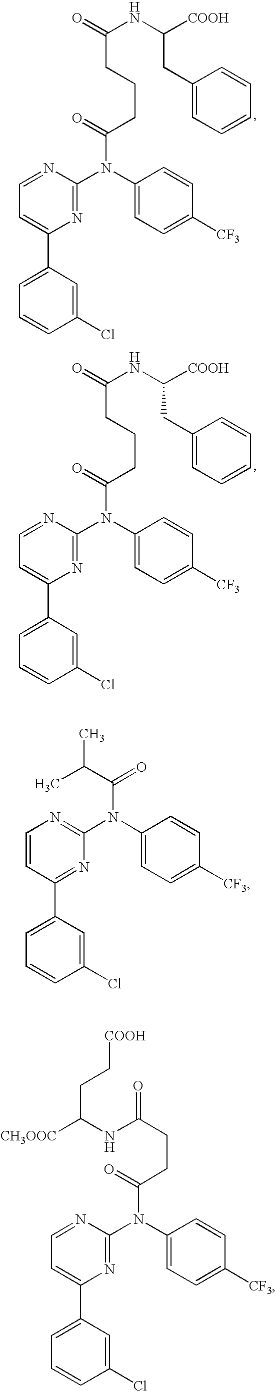

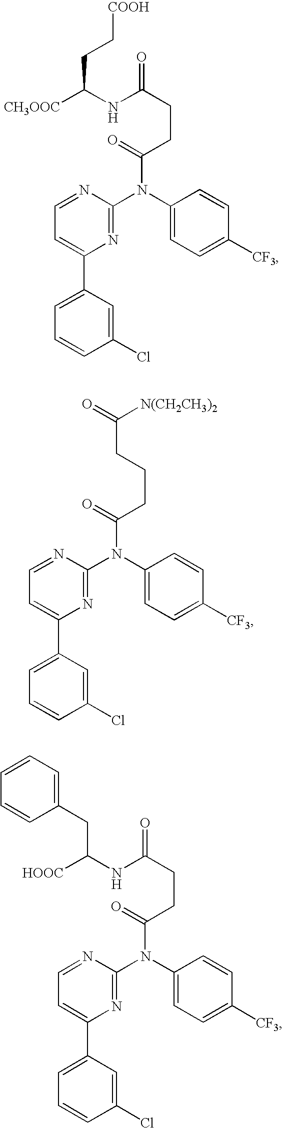

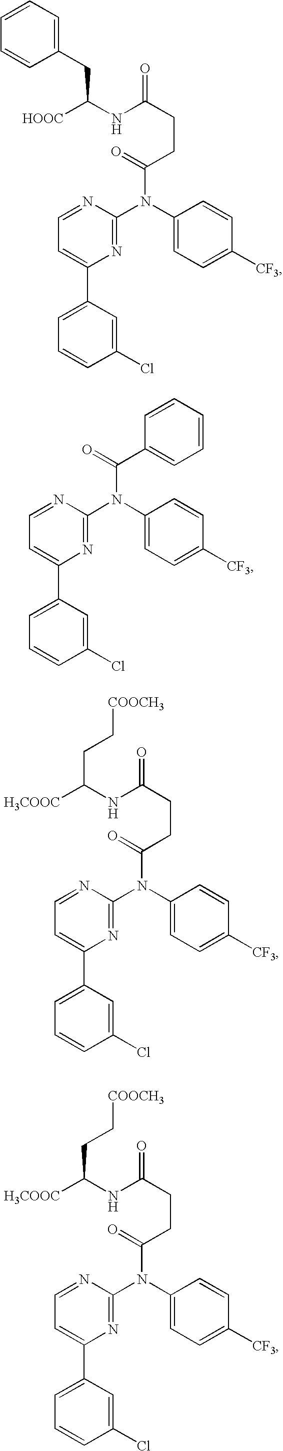

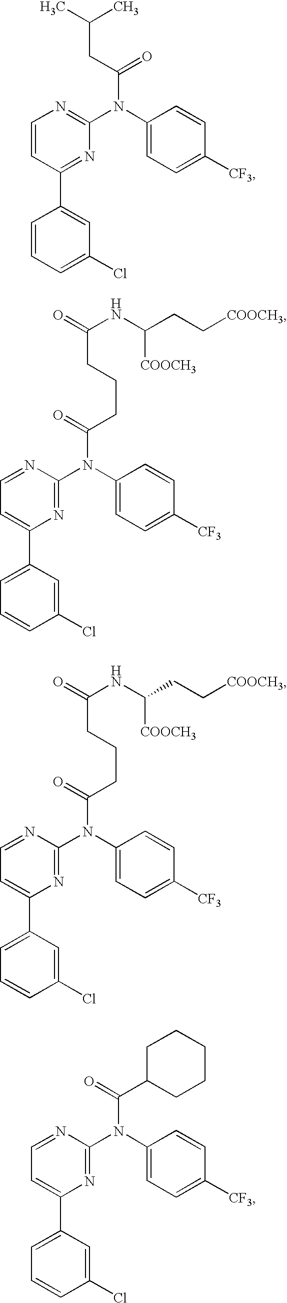

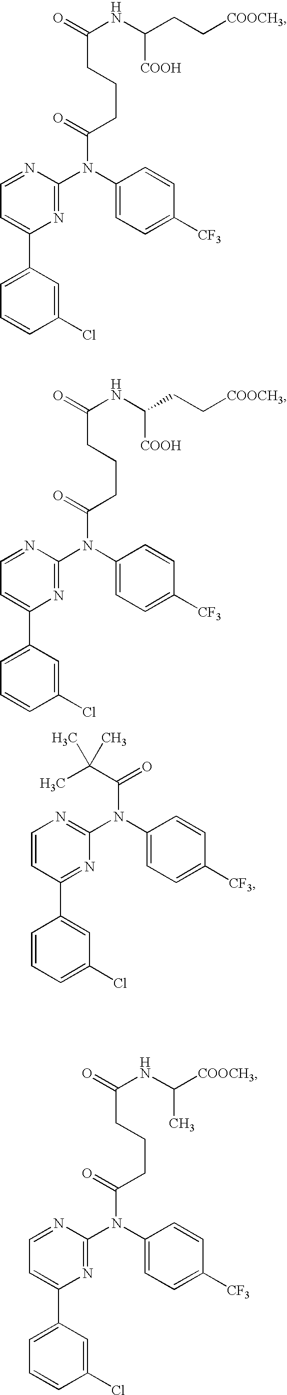

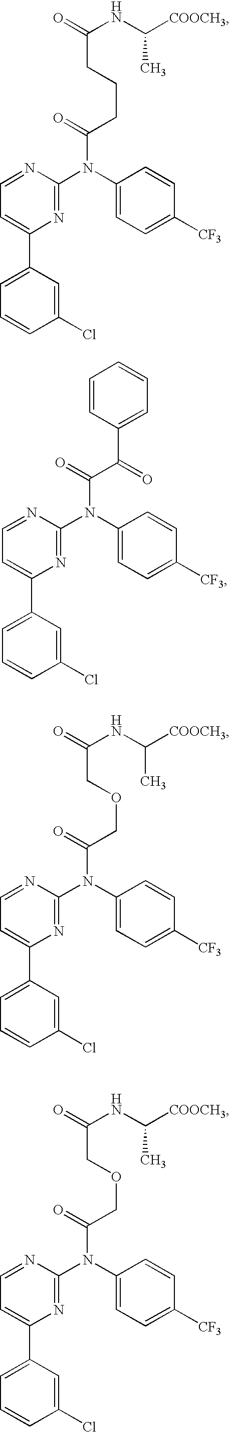

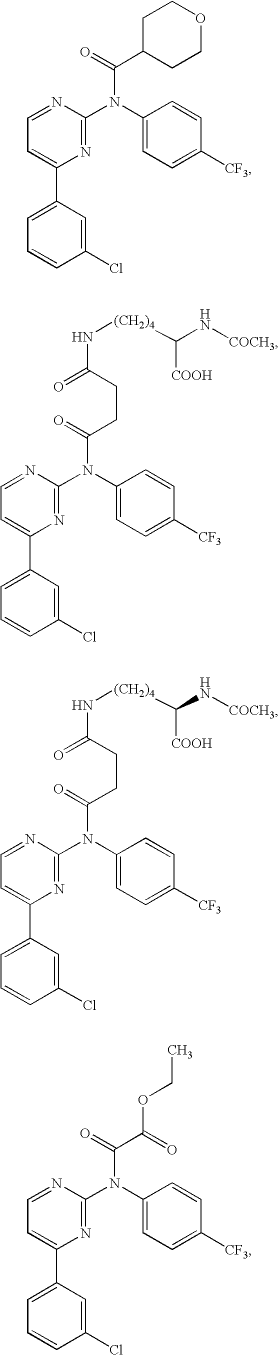

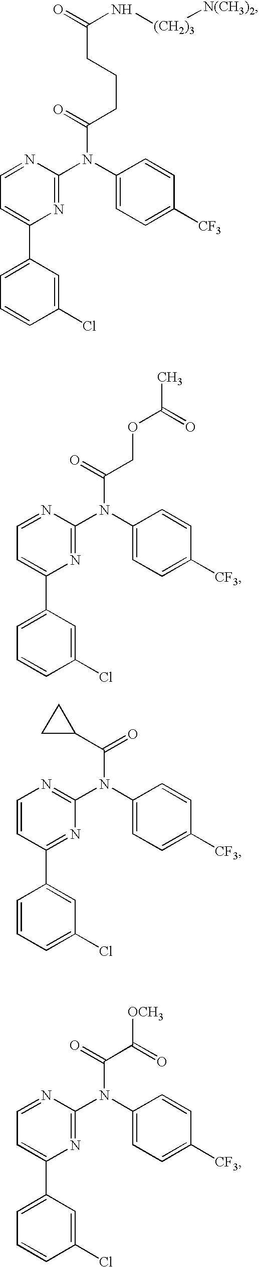

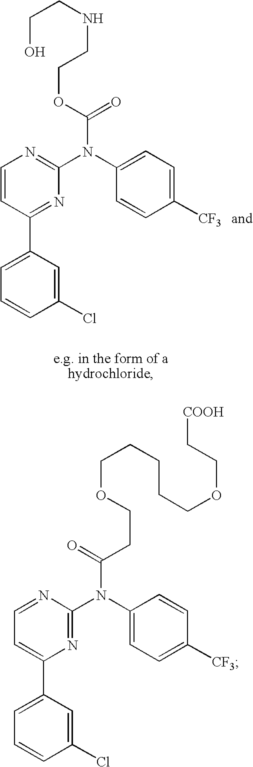

such as a compound of formula I, wherein R₁ is chloro, R₂ is hydrogen, R₃ is trifluoromethyl and R₄ is hydrogen, herein also designated as “Compound B”, or
a compound of formula I, wherein R₁ is chloro, R₂ is hydrogen, R₃ is trifluoromethyl and R₄ is a group of formula

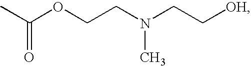

herein also designated as “Compound A”, e.g. in the form of a hydrochloride, mesylate, sulfate, tartrate, p-toluenesulfonat, besylate (monobenzenesulfonate) or calcium salt.

If not otherwise defined herein aryl includes phenyl. Halogen includes fluoro, chloro, bromo.

Haloalkyl includes halo(C₁₋₄)alkyl, wherein halo is one or more halogen, preferably trifluoromethyl. (C₃₋₈)cycloalkyl includes e.g. (C₃₋₆)cycloalkyl. Amino includes amino, (C₁₋₄)alkylamino and di(C₁₋₄)alkylamino. Aminoalkyl includes amino(C₁₋₆)alkyl, e.g. (C₁₋₄)alkylamino(C₁₋₆)alkyl, di(C₁₋₄)alkylamino(C₁₋₆)alkyl, preferably disubstituted amino(C₁₋₄)alkylamino(C₁₋₄)alkyl, e.g. dimethyl- or diethylamino(C₁₋₄)alkyl. Hydroxyalkylamino includes hydroxy(C₁₋₄)alkyl, hydroxy(C₁₋₄)alkylamino(C₁₋₄)alkyl, preferably hydroxy(C₁₋₃)alkyl or hydroxy(C₁₋₂)alkylamino(C₁₋₂)alkyl. Amino acid includes all natural and synthetic amino acids, preferably α-amino acids, e.g. alanine, phenylalanine, glycine, glutamic acid and lysine. Amino acid includes one or more of amino acid, e.g. di- or tripeptides.

Heterocyclyl includes 5 or 6 membered heterocyclic ring systems having 1 to 4 heteroatoms selected from N, O or S. Preferably the heterocyclyl is a 5 or 6 membered ring system having 1 or 2 heteroatoms selected from N or O. Preferred is pyrrolidine, morpholine and piperazine.
Any group may be unsubstituted or substituted, e.g. substituted by groups as conventional in organic chemistry, e.g. including groups selected from halogen, haloalkyl, alkylcarbonyloxy, alkoxy, hydroxy, amino, alkylcarbonylamino, aminoalkylcarbonylamino, hydroxyalkylamino, aminoalkylamino, alkylamino, dialkylamino, heterocyclyl having 5 or 6 ring members and 1 to 4 heteroatoms selected from N, O, S; (C₁₋₄)alkylheterocyclyl, wherein heterocyclyl having 5 or 6 ring members and 1 to 4 heteroatoms selected from N, O, S; hydroxy(C₁₋₄)alkylheterocyclyl, wherein heterocyclyl having 5 or 6 ring members and 1 to 4 heteroatoms selected from N, O, S; carboxyl, (C₁₋₄)alkylcarbonyloxy, amino(C₁₋₄)-alkylcarbonyloxy.

A phenyl-(4-phenyl-pyrimidin-2-yl)-amine according to the present invention includes a phenyl-(4-phenyl-pyrimidin-2-yl)-amine in any form, e.g. in free form, in the form of a salt, in the form of a solvate and in the form of a salt and a solvate. Such salts include a pharmaceutically acceptable salt, e.g. including a metal salt or an acid addition salt. Metal salts include for example alkali or earth alkali salts; such as a calcium salt. Acid addition salts include salts of a compound of formula I with an acid, e.g. including inorganic and organic acids, e.g. including pharmaceutically acceptable acids, such as hydrochloric acid, sulfuric acid, methanesulfonic acid, benzenesulfonic acid, p-toluenesulfonic acid, tartaric acid.

A phenyl-(4-phenyl-pyrimidin-2-yl)-amine according to the present invention in free form may be converted into a corresponding compound in the form of a salt; and vice versa. A phenyl-(4-phenyl-pyrimidin-2-yl)-amine according to the present invention in free form or in the form of a salt and in the form of a solvate may be converted into a corresponding compound in free form or in the form of a salt in a non-solvate form; and vice versa.

A phenyl-(4-phenyl-pyrimidin-2-yl)-amine according to the present invention may exist in the form of isomers and mixtures thereof; e.g. optical isomers, diastereoisomers, cis-trans conformers. A phenyl-(4-phenyl-pyrimidin-2-yl)-amine according to the present invention may e.g. contain asymmetric carbon atoms and may thus exist in the form of enantiomeres, diastereoisomers and mixtures thereof, e.g. racemates. E.g. a substitutent attached to an asymmetric carbon atom in a phenyl-(4-phenyl-pyrimidin-2-yl)-amine according to the present invention may be in the R- or in the S-configuration, including mixtures thereof. The present invention includes a phenyl-(4-phenyl-pyrimidin-2-yl)-amine according to the present invention in any isomeric form and in any isomeric mixture.

A phenyl-(4-phenyl-pyrimidin-2-yl)-amine according to the present invention may be prepared as appropriate, e.g. according, e.g. analogously to a process as convential, e.g. a compound of formula I may be e.g. prepared according, e.g. analogously, to a method as disclosed in WO03063871.

The pharmaceutical utility of a phenyl-(4-phenyl-pyrimidin-2-yl)-amine according to the present invention e.g. may be determined in an animal model, e.g. the in vivo effects of a phenyl-(4-phenyl-pyrimidin-2-yl)-amine according to the present invention have been determined in a murine model of allogeneic pancreatic islets transplantation. The compounds of the present invention are active in such model.

E.g. the effects of a Compound A and a Compound B, herein also designated as “TEST COMPOUND” (abbreviated also as “TC”, e.g. in the Figures), in such murine model of allogeneic pancreatic islets transplantation has been investigated, e.g. by the following procedures:
Recipient BALB/c mice are rendered diabetic by a single dose of streptozotocin and are transplanted with pancreatic islets isolated from C57Bl/6 mice. Transplanted mice are treated daily with a water-soluble derivative of a TEST COMPOUND from day 0 until 30 days post transplantation at a dose of 30 mg/kg or 7.5 mg/kg. A TEST COMPOUND was found to significantly promoting long-term pancreatic islets allograft acceptance in a dose dependent manner. E.g., at a dosage of 7.5 mg/kg, the TEST COMPOUND induces long-term graft survival in 40% of the recipients, whereas at 30 mg/kg, 70% of treated mice maintain normal glucose levels (see e.g. results in FIG. 1). Interestingly, the treatment with the higher TEST COMPOUND dose results in the induction of active tolerance, as demonstrated by re-challenging long-term tolerant mice at day 100 with donor type spleenocytes (see e.g. results in FIG. 2). In addition, antigen presenting cells isolated from spleens of TEST-COMPOUND-treated mice with long-term graft survival transfer tolerance into newly transplanted mice (see e.g. results in FIG. 3). Ex vivo flow cytometric analysis performed 7 and 30 days post transplantation reveals that treatment with TEST COMPOUND results in down-regulation of the expression of costimulatory molecules (CD80, CD86, CD40) on CD11c+ splenic DCs (see e.g. results in FIG. 4). Histological analysis of the transplanted islets shows that TEST COMPOUND treatment inhibits graft infiltration by CD11c+ DCs and CD8+ T cells, and preserves islet function (FIG. 5). These findings indicate that TEST COMPOUND is a tolerogenic compound that specifically inhibits activation and function or DCs.

To further investigate the effect of TEST COMPOUND on murine DCs bone marrow cells from BALB/c mice are differentiated with GM-CSF into bone marrow derived DCs (BM-DCs) in the presence or absence of TEST COMPOUND (20 nM). BM-DC are left immature or activated with LPS. TEST COMPOUND inhibits the up-regulation of costimulatory molecules, CD80, CD86, and CD40, on mature BM-DC (see e.g. results in FIG. 6). Interestingly, injection of LPS-activated, but not immature TEST-COMPOUND-treated recipient type BM-DC into mice significantly promotes islets allograft acceptance (see e.g. results in FIG. 7).

A method or a pharmaceutical composition or the use according to the present invention is thus shown to be useful for treating or preventing disorders or disease states wherein low immunotolerance is a basis for a disorder or disease state.

A method or a pharmaceutical composition or the use according to the present invention, e.g. such as indicated under 1.1 to 1.4, 2.1 to 2.3 and 4.1 above, may be useful for the treatment of disorders, e.g. including treating diseases, which are mediated by low immunotolerance, e.g. including
a) Treatment of disorders following organ or tissue transplant rejection, e.g. for the treatment of recipients of e.g. heart, lung, combined heart-lung, liver, kidney, pancreatic, skin or corneal transplants; for the prevention of graft-versus-host disease, such as following bone marrow transplantation, e.g. including chronic rejection in a transplanted organ, such as manifestations of chronic rejection, namely conditions resulting from the immune reaction towards the graft and the response of the blood vessel walls in the grafted organ or tissue. A method or the use according to the present invention may be useful for reducing chronic rejection manifestations or for ameliorating the conditions resulting from chronic rejection. The organ or tissue transplantation may be performed from a donor to a recipient of a same or different species. A method or the use according to the present invention may thus be useful also for preventing or combating acute or chronic rejection in a recipient of organ or tissue xenograft transplant. Transplanted organs, e.g. including xenograft organ or tissue transplants, may include e.g. heart, liver, kidney, spleen, lung, small bowel, pancreatic (complete or partial, e.g. Langerhans islets), skin and bone marrow xenografts.
b) Treatment of autoimmune disorders and of (malignant) inflammatory conditions, in particular inflammatory conditions with an etiology including an autoimmune component, such as arthritis (for example rheumatoid arthritis, arthritis chronica progrediente and arthritis deformans) and rheumatic diseases. Specific autoimmune diseases for which a method or the use according to the present invention may be useful include, autoimmune hematological disorders (including e.g. hemolytic anemia, aplastic anaemia, pure red cell anaemia and idiopathic thrombocytopenia), systemic lupus erythematosus, polychondritis, scleredoma, Wegener granulomatosis, dermatomyositis, chronic active hepatitis, myasthenia gravis, psoriasis, Steven-Johnson syndrome, idiopathic sprue, gastrointestinal diseases, such as autoimmune inflammatory bowel disease (including e.g. ulcerative colitis and Crohn's disease) endocrine ophthalmopathy, Graves disease, sarcoidosis, multiple sclerosis, primary billiary cirrhosis, juvenile diabetes (diabetes mellitus type I), uveitis (anterior and posterior), keratoconjunctivitis sicca and vernal keratoconjunctivitis, interstitial lung fibrosis, psoriatic arthritis, glomerulonephritis (with and without nephrotic syndrome, e.g. including idiopathic nephrotic syndrome or minimal change nephropathy) and juvenile dermatomyositis.
c) Treatment of allergic disorders, e.g. allergic conditions with an etiology including an autoimmune component, such as atopic dermatitis, particularly in children, urticaria, particularly acute urticaria, allergic asthma, allergic rhinitis, food allergies, allergic conjunctivitis, hayfever, bullous pemphigoid, industrial sensitization.
   Treatment as used herein includes treatment and prophylaxis (prevention).

In another aspect the present invention provides the use, a method or a pharmaceutical composition according to the present invention e.g. such as indicated under 1.1 to 1.4, 2.1 to 2.3 and 4.1 above, for the treatment of disorders mediated by low immunotolerance, e.g. including
disorders following organ or tissue transplant rejection,
autoimmune disorders,
inflammatory conditions with an etiology including an autoimmune component,
allergic disorders such as
A method of treating disorders mediated by low immunotolerance comprising administering to a subject in need of such treatment a therapeutically effective amount of a phenyl-(4-phenyl-pyrimidin-2-yl)-amine according to the present invention;
A method for the preparation of a medicament for treating disorders mediated by low immunotolerance comprising a phenyl-(4-phenyl-pyrimidin-2-yl)-amine according to the present invention; e.g. in an effective amount;
The use of a pharmaceutical composition of the present invention for treating disorders mediated by low immunotolerance, or
A pharmaceutical composition for treating disorders mediated by low immunotolerance comprising a pharmaceutical composition according to the present invention.

In a method or a use according to the present invention a phenyl-(4-phenyl-pyrimidin-2-yl)-amine according to the present invention may be used as the sole active ingredient, or at least one second drug substance may be used in combination, e.g. either in sequence, or concomitantly, e.g. a second drug substance, as defined herein, for treating disorders mediated by low immunotolerance.

Such second drug substance e.g. include immunomodulatory compounds, such as immunosuppressants, anti-inflammatory compounds or anti-allergic compounds. A pharmaceutical composition according to the present invention optionally may comprise a combination of mature dendritic cells which are matured outside of the mammalian body in the presence of a phenyl-(4-phenyl-pyrimidin-2-yl)-amine and a second drug substance, preferably a pharmaceutical composition according to the present invention does not comprise a second drug substance.

For treatment according to the present invention, the appropriate dosage of the second drug substance will, of course, vary depending upon, for example, the chemical nature and the pharmacokinetic data of the second drug substance used, the individual host, the mode of administration and the nature and severity of the conditions being treated. However, in general, for satisfactory results in larger mammals, for example humans, an indicated daily dosage includes a range of
from about 0.0001 g to about 1.5 g,
from about 0.01 mg/kg body weight to about 20 mg/kg body weight,
   for example administered in divided doses up to four times a day.

A second drug substance in a combination (treatment) according to the present invention may be administered by any conventional route, for example enterally, e.g. including nasal, buccal, rectal, oral, administration; parenterally, e.g. including intravenous, intraarterial, intramuscular, intracardiac, subcutanous, intraosseous infusion, transdermal (diffusion through the intact skin), transmucosal (diffusion through a mucous membrane), inhalational administration; topically; e.g. including epicutaneous, intranasal, intratracheal administration; intraperitoneal (infusion or injection into the peritoneal cavity); epidural (peridural) (injection or infusion into the epidural space); intrathecal (injection or infusion into the cerebrospinal fluid); intravitreal (administration via the eye); or via medical devices, e.g. for local delivery, e.g. stents,

e.g. in form of coated or uncoated tablets, capsules, (injectable) solutions, infusion solutions, solid solutions, suspensions, dispersions, solid dispersions; e.g. in the form of ampoules, vials, in the form of creams, gels, pastes, inhaler powder, foams, tinctures, lip sticks, drops, sprays, or in the form of suppositories.
A second drug substance may be administered in a combination treatment according to the present invention the form of a pharmaceutically acceptable salt, or in free form; optionally in the form of a solvate.

In a series of further aspects the present invention provides
A combination of a pharmaceutical composition of the present invention together with at least one second drug substance;
A pharmaceutical combination comprising a pharmaceutical composition according the present invention in combination with at least one second drug substance;
A pharmaceutical composition according the present invention in combination with at least one second drug substance, for use in any method as defined herein,
A pharmaceutical combination comprising a pharmaceutical composition according the present invention and at least one second drug substance for use as a pharmaceutical, e.g. for use in combination treatment;
The use as a pharmaceutical of a pharmaceutical composition according the present invention in combination with at least one second drug substance, e.g. for use in combination treatment;
The use of a pharmaceutical composition according the present invention for the manufacture of a medicament for use in combination with a second drug substance;
The use of a pharmaceutical composition according the present invention for the manufacture of a medicament for use in combination with a second drug substance, for the treatment of disorders mediated by low immunotolerance,
A method for treating disorders mediated by low immunotolerance in a subject in need thereof, comprising co-administering, concomitantly or in sequence, a therapeutically effective amount of a pharmaceutical composition according to the present invention and at least one second drug substance, e.g. in the form of a pharmaceutical combination or composition;
A pharmaceutical composition according to the present invention in combination with at least one second drug substance for use in the preparation of a medicament for use for the treatment of disorders mediated by low immunotolerance.

Combinations include free combinations in which a pharmaceutical composition of the present invention and at least one second drug substance are packaged separately, but instruction for concomitant or sequential administration are given, e.g. for the treatment of disorders mediated by low immunotolerance.

In another aspect the present invention provides a pharmaceutical package comprising at least one second drug substance beside instructions for combined administration with a pharmaceutical composition of the present invention, e.g. instructions for combination treatment.

Combination treatment according to the present invention may provide improvements compared with single treatment.

In another aspect the present invention provides
A method for improving the therapeutic utility of a pharmaceutical composition of the present invention comprising co-administering, e.g. concomitantly or in sequence, of a therapeutically effective amount of a compound of the present invention and a second drug substance.
A method for improving the therapeutic utility of a second drug substance comprising co-administering, e.g. concomitantly or in sequence, of a therapeutically effective amount of a pharmaceutical composition of the present invention and a second drug substance.

Pharmaceutical compositions according to the present invention may be manufactured according, e.g. analogously, to a method as conventional, e.g. by mixing, granulating, coating, dissolving or lyophilizing processes. Unit dosage forms may contain, for example, from about 0.1 ma to about 1500 mg, such as 1 mg to about 1000 mg.

By the term “second drug substance” is meant a chemotherapeutic drug, especially any chemotherapeutic agent other than a. phenyl-(4-phenyl-pyrimidin-2-yl)-amine according to the present invention.

For example, a second drug substance as used herein includes e.g. an anti-inflammatory and/or immunomodulatory and/or anti-allergic drug.

Anti-inflammatory and/or immunomodulatory and/or anti-allergic drugs which are prone to be useful in combination with a compound of the present invention include e.g.
mediators, e.g. inhibitors, of mTOR activity, including rapamycin of formula

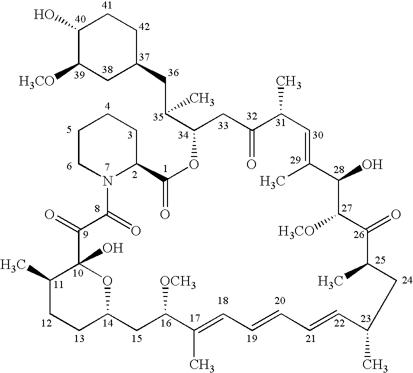

and rapamycin derivatives, e.g. including
40-O-alkyl-rapamycin derivatives, such as 40-O-hydroxyalkyl-rapamycin derivatives, such as 40-O-(2-hydroxy)-ethyl-rapamycin (everolimus),
32-deoxo-rapamycin derivatives and 32-hydroxy-rapamycin derivatives, such as 32-deoxorapamycin,
16-O-substituted rapamycin derivatives such as 16-pent-2-ynyloxy-32-deoxorapamycin, 16-pent-2-ynyloxy-32 (S or R)-dihydro-rapamycin, 16-pent-2-ynyloxy-32 (S or R)-dihydro-40-O-(2-hydroxyethyl)-rapamycin,
rapamycin derivatives which are acylated at the oxygen group in position 40, e.g. 40-[3-hydroxy-2-(hydroxy-methyl)-2-methylpropanoate]-rapamycin (also known as CCI779), rapamycin derivatives which are substituted in 40 position by heterocyclyl, e.g. 40-epi-(tetrazolyl)-rapamycin (also known as ABT578),
the so-called rapalogs, e.g. as disclosed in WO9802441, WO0114387 and WO0364383, such as AP23573, and
compounds disclosed under the name TAFA-93, AP23464, AP23675, AP23841 and biolimus (e.g. biolimus A9).
mediators, e.g. inhibitors, of calcineurin, e.g. cyclosporin A, FK 506;
ascomycins having immuno-suppressive properties, e.g. ABT-281, ASM981;
corticosteroids; cyclophosphamide; azathioprene; leflunomide; mizoribine;
mycophenolic acid or salt, e.g. sodium; mycophenolate mofetil;
15-deoxyspergualine or an immunosuppressive homologue, analogue or derivative thereof;
mediators, e.g. inhibitors, of bcr-abl tyrosine kinase activity;
mediators, e.g. inhibitors, of c-kit receptor tyrosine kinase activity;
mediators, e.g. inhibitors, of PDGF receptor tyrosine kinase activity, e.g. Gleevec (imatinib);
mediators, e.g. inhibitors, of p38 MAP kinase activity,
mediators, e.g. inhibitors, of VEGF receptor tyrosine kinase activity,
mediators, e.g. inhibitors, of PKC activity, e.g. as disclosed in WO0238561 or WO0382859, e.g. the compound of Example 56 or 70;
mediators, e.g. inhibitors, of JAK3 kinase activity, e.g. N-benzyl-3,4-dihydroxy-benzylidene-cyanoacetamide □-cyano-(3,4-dihydroxy)-]N-benzylcinnamamide (Tyrphostin AG 490), prodigiosin 25-C (PNU 156804), [4-(4′-hydroxyphenyl)-amino-6,7-dimethoxyquinazoline] (WHI-P131), [4-(3′-bromo-4′-hydroxylphenyl)-amino-6,7-dimethoxyquinazoline] (WHI-P154), [4-(3′,5′-dibromo-4′-hydroxylphenyl)-amino-6,7-dimethoxyquinazoline] WHI-P97, KRX-211, 3-{(3R,4R)-4-methyl-3-[methyl-(7H-pyrrolo[2,3-d]pyrimidin-4-yl)-amino]-piperidin-1-yl}-3-oxo-propionitrile, in free form or in a pharmaceutically acceptable salt form, e.g. mono-citrate (also called CP-690,550), or a compound as disclosed in WO2004052359 or WO2005066156;
mediators, e.g. agonists or modulators of S1P receptor activity, e.g. FTY720 optionally phosphorylated or an analog thereof, e.g. 2-amino-2-[4-(3-benzyloxyphenylthio)-2-chlorophenyl]ethyl-1,3-propanediol optionally phosphorylated or 1-{4-[1-(4-cyclohexyl-3-trifluoromethyl-benzyloxyimino)-ethyl]-2-ethyl-benzyl}-azetidine-3-carboxylic acid or its pharmaceutically acceptable salts;
immunosuppressive monoclonal antibodies, e.g., monoclonal antibodies to leukocyte receptors, e.g., Blys/BAFF receptor, MHC, CD2, CD3, CD4, CD7, CD8, CD25, CD28, CD40, CD45, CD52, CD58, CD80, CD86, IL-12 receptor, IL-17 receptor, IL-23 receptor or their ligands;
other immunomodulatory compounds, e.g. a recombinant binding molecule having at least a portion of the extracellular domain of CTLA4 or a mutant thereof, e.g. an at least extracellular portion of CTLA4 or a mutant thereof joined to a non-CTLA4 protein sequence, e.g. CTLA4Ig (for ex. designated ATCC 68629) or a mutant thereof, e.g. LEA29Y;
mediators, e.g. inhibitors of adhesion molecule activities, e.g. LFA-1 antagonists, ICAM-1 or -3 antagonists, VCAM-4 antagonists or VLA-4 antagonists,
mediators, e.g. antagonists of CCR9 activity,
mediators, e.g. inhibitors, of MIF activity,
5-aminosalicylate (5-ASA) agents, such as sulfasalazine, Azulfidine®), Asacol®, Dipentum®), Pentasa®, Rowasa®, Canasa®, Colazal®, e.g. drugs containing mesalamine; e.g mesalazine in combination with heparin;
mediators, e.g. inhibitors, of TNF-alpha activity, e.g. including antibodies which bind to TNF-alpha, e.g. infliximab (Remicade®), thalidomide, lenalidomide,
nitric oxide releasing non-steriodal anti-inflammatory drugs (NSAIDs), e.g. including COX-inhibiting NO-donating drugs (CINOD);
phosphordiesterase, e.g. mediators such as inhibitors of PDE4B activity,
mediators, e.g. inhibitors, of caspase activity,
mediators, e.g. agonists, of the G protein coupled receptor GPBAR1,
mediators, e.g. inhibitors, of ceramide kinase activity,
‘multi-functional anti-inflammatory’ drugs (MFAIDs), e.g. cytosolic phoshpholipase A2 (cPLA2) inhibitors, such as membrane-anchored phospholipase A2 inhibitors linked to glycosaminoglycans;
antibiotics, such as penicillins, cephalosporins, erythromycins, tetracyclines, sulfonamides, such as sulfadiazine, sulfisoxazole; sulfones, such as dapsone; pleuromutilins, fluoroquinolones, e.g. metronidazole, quinolones such as ciprofloxacin; levofloxacin; probiotics and commensal bacteria e.g. Lactobacillus, Lactobacillus reuteri;
antiviral drugs, such as ribivirin, vidarabine, acyclovir, ganciclovir, zanamivir, oseltamivir phosphate, famciclovir, atazanavir, amantadine, didanosine, efavirenz, foscarnet, indinavir, lamivudine, nelfinavir, ritonavir, saquinavir, stavudine, valacyclovir, valganciclovir, zidovudine.

Anti-inflammatory which are prone to be useful in combination with a compound of the present invention include e.g. non-steroidal antiinflammatory agents (NSAIDs) such as propionic acid derivatives (alminoprofen, benoxaprofen, bucloxic acid, carprofen, fenbufen, fenoprofen, fluprofen, flurbiprofen, ibuprofen, indoprofen, ketoprofen, miroprofen, naproxen, oxaprozin, pirprofen, pranoprofen, suprofen, tiaprofenic acid, and tioxaprofen), acetic acid derivatives (indomethacin, acemetacin, alclofenac, clidanac, diclofenac, fenclofenac, fenclozic acid, fentiazac, furofenac, ibufenac, isoxepac, oxpinac, sulindac, tiopinac, tolmetin, zidometacin, and zomepirac), fenamic acid derivatives (flufenamic acid, meclofenamic acid, mefenamic acid, niflumic acid and tolfenamic acid), biphenylcarboxylic acid derivatives (diflunisal and flufenisal), oxicams (isoxicam, piroxicam, sudoxicam and tenoxican), salicylates (acetyl salicylic acid, sulfasalazine) and the pyrazolones (apazone, bezpiperylon, feprazone, mofebutazone, oxyphenbutazone, phenylbutazone); cyclooxygenase-2 (COX-2) inhibitors such as celecoxib; inhibitors of phosphodiesterase type IV (PDE-IV); antagonists of the chemokine receptors, especially CCR-1, CCR-2, and CCR-3; cholesterol lowering agents such as HMG-CoA reductase inhibitors (lovastatin, simvastatin and pravastatin, fluvastatin, atorvastatin, and other statins), sequestrants (cholestyramine and colestipol), nicotinic acid, fenofibric acid derivatives (gemfibrozil, clofibrat, fenofibrate and benzafibrate), and probucol; anticholinergic agents such as muscarinic antagonists (ipratropium bromide); other compounds such as theophylline, sulfasalazine and aminosalicylates, e.g. 5-aminosalicylic acid and prodrugs thereof, antirheumatics.

Antiallergic drugs which are prone to be useful in combination with a compound of the present invention include e.g. antihistamines (Hi-histamine antagonists), e.g. bromopheniramine, chlorpheniramine, dexchlorpheniramine, triprolidine, clemastine, diphenhydramine, diphenylpyraline, tripelennamine, hydroxyzine, methdilazine, promethazine, trimeprazine, azatadine, cyproheptadine, antazoline, pheniramine pyrilamine, astemizole, terfenadine, loratadine, cetirizine, fexofenadine, descarboethoxyloratadine, and non-steroidal anti-asthmatics such as β2-agonists (terbutaline, metaproterenol, fenoterol, isoetharine, albuterol, bitolterol, salmeterol and pirbuterol), theophylline, cromolyn sodium, atropine, ipratropium bromide, leukotriene antagonists (zafirlukast, montelukast, praniukast, iralukast, pobilukast, SKB-106,203), leukotriene biosynthesis inhibitors (zileuton, BAY-1005); bronchodilators, antiasthmatics (mast cell stabilizers).

Preferably a second drug includes immunomodulators, e.g.
mediators, e.g. inhibitors, of mTOR activity,
mediators, e.g. inhibitors, of calcineurin,
ascomycins having immunosuppressive properties,
corticosteroids; cyclophosphamide; azathioprene; leflunomide; mizoribine;
mycophenolic acid or salt, e.g. sodium; mycophenolate mofetil;
15-deoxyspergualine or an immunosuppressive homologue, analogue or derivative thereof;
mediators, e.g. agonists or modulators of S1P receptor activity,
immunosuppressive monoclonal antibodies;
a recombinant binding molecule having at least a portion of the extracellular domain of CTLA4 or a mutant thereof, e.g. an at least extracellular portion of CTLA4 or a mutant thereof joined to a non-CTLA4 protein sequence,
mediators, e.g. inhibitors of adhesion molecule activities, e.g. LFA-1 antagonists,
mediators, e.g. antagonists of CCR9 activity,
mediators, e.g. inhibitors, of MIF activity,
5-aminosalicylate (5-ASA) agents,
mediators, e.g. inhibitors, of TNF-alpha activity,
nitric oxide releasing non-steriodal anti-inflammatory drugs (NSAIDs), e.g. including COX-inhibiting NO-donating drugs (CINOD);
mediators, e.g. agonists, of the G protein coupled receptor GPBAR1,
mediators, e.g. inhibitors, of ceramide kinase activity,
‘multi-functional anti-inflammatory’ drugs (MFAIDs),
antibiotics,

### DESCRIPTION OF THE FIGURES

In all FIGS. 1 to 7, “TC” is the TEST COMPOUND, which test compound is as described above. For oral administration Compound A is used, for cell treatment either Compound A or Compound B may be used, preferably compound A is also used for cell treatment.

FIGS. 1 to 3

### Show the Induction of Transferable Active Tolerance by the TEST COMPOUND (TC)

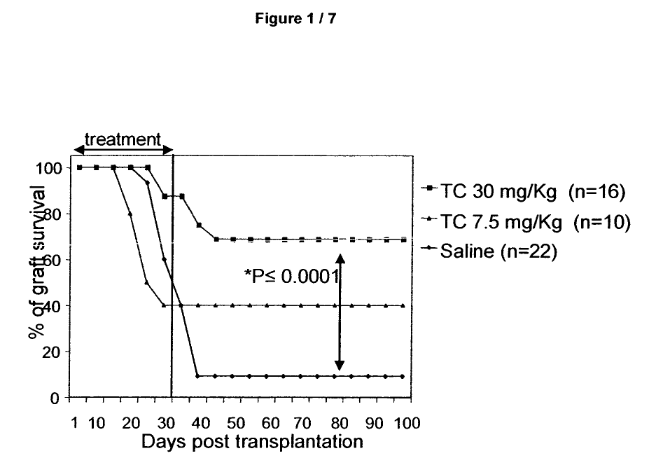

FIG. 1

BALB/c female mice transplanted with pancreatic islets isolated from C57BI/6 allogeneic donors are treated with the TEST COMPOUND from day 0 to 30 post transplantation (at 30 or 7.5 mg/kg/day). Graft survival is monitored by weekly measurement of blood glucose level. At 7.5 mg/kg, the TEST COMPOUND induces long-term graft survival in 40% of the recipients, whereas at 30 mg/kg, 70% of treated mice maintain normal glucose levels.

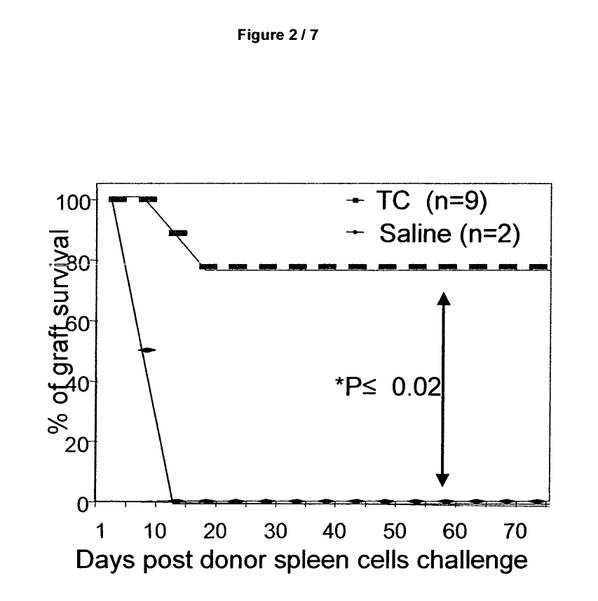

FIG. 2

TEST COMPOUND (30 mg/kg/day) treated and control mice with long-term graft survival, as shown in FIG. 1, are challenged at day 100 with donor splenocytes (3×107 i.p.). The treatment with the higher TEST COMPOUND dose results in the induction of active tolerance.

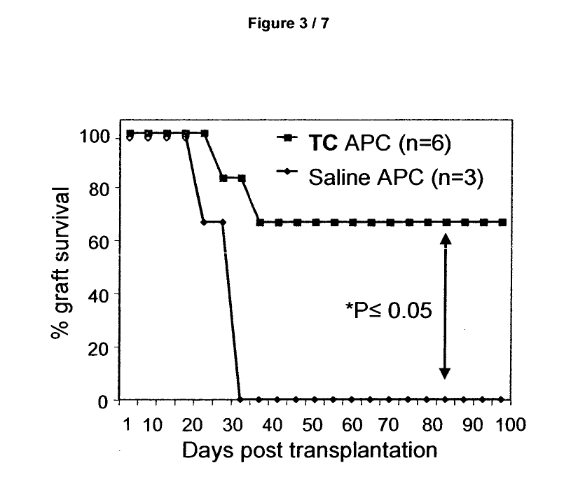

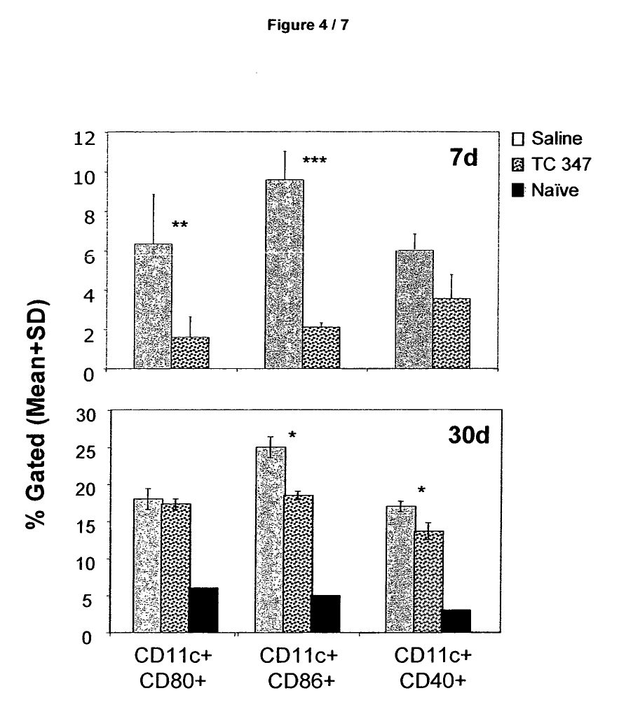

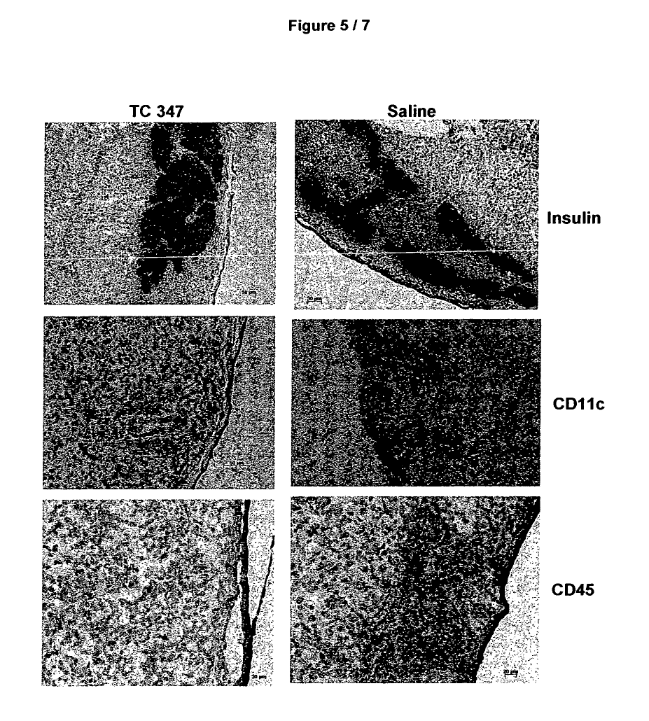

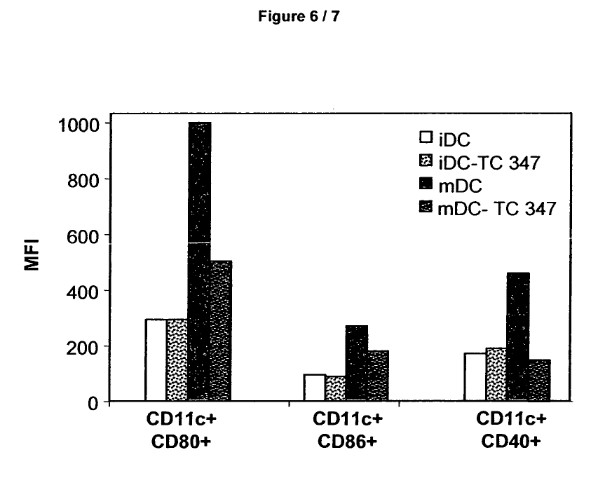

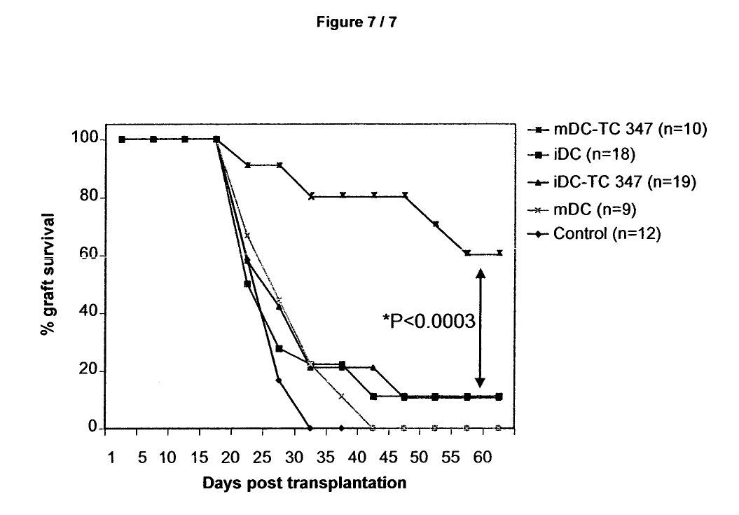

FIG. 3

Splenocytes from TEST-COMPOUND-treated mice (30 mg/kg/day) that accepted islet allografts are depleted of CD90 (Thy1)-expressing cells and the remaining APC population is injected (5×106 cells, i.v.) into newly transplanted recipients. Graft survival is monitored by weekly measurement of blood glucose level (*two-tailed t-test). APCs isolated from spleens of TEST-COMPOUND-treated mice with long-term graft survival transfer tolerance into newly transplanted mice.

FIG. 4

### Shows Lower Frequency of Activated DCs in Spleens of TESTCOMPOUND-Treated Mice

Spleen cells from islet allograft transplanted mice7d or 30d (n=3 per group per time point) post transplantation are analyzed by FACS for the expression of the indicated activation markers by CD11c+DCs. (2-tailed t-tests: ***P<0.001, **P<0.01, *P<0.05). Treatment with TEST COMPOUND results in down-regulation of the expression of costimulatory molecules (CD80, CD86, CD40) on CD11c+splenic DCs.

FIG. 5

Shows Inhibition of Immune Cell Infiltration into Islet Allografts of TEST-COMPOUND-Treated Mice

BALB/c recipient mice are transplanted with C57BI/6 islets and treated orally with saline or TEST COMPOUND (30 mg/kg/day). Ten days after transplantation, kidneys are extracted, cryosectioned at the transplant area and stained for the indicated markers.

Histological analysis of the transplanted islets shows that TEST COMPOUND treatment inhibits graft infiltration by CD11c+DCs and CD8+ T cells, and preserves islet function.

FIGS. 6 and 7

Show Cellular Therapy with In Vitro TEST COMPOUND-Treated BMDC

FIG. 6

BALB/c BM-DC are differentiated from bone-marrow cells cultured with GM-CSF (20 ng/ml), in the presence or absence of TEST COMPOUND (20 nM). BM-DC are left immature (iDC) or activated with LPS (1 mg/ml, mDC). DCs are harvested and stained for the indicated markers. TEST COMPOUND inhibits the up-regulation of costimulatory molecules, CD80, CD86, and CD40, on mature BM-DC.

FIG. 7

BM-DC are injected (3×105, i.v.) into BALB/c diabetic female mice one day before C57BI/6 islet allograft transplantation. Graft survival is monitored by weekly measurement of blood glucose level (*two-tailed t test). Injection of LPS-activated, but not immature TEST-COMPOUND-treated recipient type BM-DC into mice significantly promotes islets allograft acceptance.

1-2. (canceled)

3. A method for enhancing or inducing immunotolerance in a mammal, comprising the steps of
a) isolating monocytes and/or dendritic cells from a mammal,
b) differentiating the monocytes, into immature dendritic cells (iDC), in the presence of an phenyl-(4-phenyl-pyrimidin-2-yl) amine,
c) treating immature dendritic cells and dendritic cells obtained in step a) or b) with a maturation agent in the presence of an phenyl-(4-phenyl-pyrimidin-2-yl) amine, and
d) administering an effective amount of maturated dendritic cells obtained in step c) to said mammal.

4. A pharmaceutical composition comprising mature dendritic cells which are matured and treated outside of the mammalian body in the presence of an phenyl-(4-phenyl-pyrimidin-2-yl)-amine, and a pharmaceutically acceptable excipient.

5. A process for the production of a pharmaceutical composition comprising
a) maturing dendritic cells outside of the mammalian body in the presence of an phenyl-(4-phenyl-pyrimidin-2-yl)-amine,
b) isolating the cells obtained in step a), and
c) mixing with pharmaceutically acceptable excipient.

6. A pharmaceutical composition according to claim 4 for the treatment of disorders mediated by low immunotolerance.

7: A pharmaceutical composition or a process for its production according to claim 4, wherein the pharmaceutical composition is adapted for intravenous administration.

8. A process for the manufacture of a medicament for the treatment of disorders mediated by low immunotolerance comprising preparing a pharmaceutical composition according to claim 4.

9. A combination of a pharmaceutical composition according to claim 4, together with at least one second drug substance.

10. A method for treating disorders mediated by low immunotolerance in a subject in need thereof, comprising administering a therapeutically effective amount of a pharmaceutical composition according to claim 4 to a subject in need thereof.

11. A method according to claim 10, further comprising a therapeutically effective amount of at least one second drug substance.

12. The method of claim 3, wherein the phenyl-(4-phenyl-pyrimidin-2-yl)-amine is a compound of formula 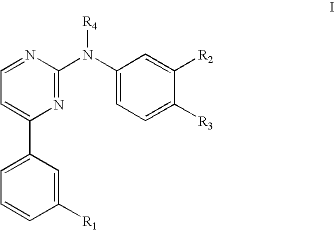
wherein
R₁ is halogen or halo(C₁₋₄)alkyl,
R₂ is hydrogen, halogen or halo(C₁₋₄)alkyl,
R₃ is halogen or halo(C₁₋₄)alkyl,
R₄ is hydrogen, (C₁₋₈)alkyl, hydroxy(C₁₋₆)alkyl or a group of formula
—CO—R₅,
—CO—(CH₂)ₘ—OR₆,
—CO—CO—R₇,
—CO—CO—OR₈,
—CON(R₉R₁₀),
—CO—(CH₂)ₙ—CO—R₁₁,
—CO—(CHR₁₅)—O(CH₂)ₒ—CO—R₁₁,
—CO—(CH₂)ₚ—O—(CH₂)_{q}—O—(CH₂)ᵣ—R₁₆,
—CO—O—(CH₂), —O—CO—R₁₇,
—CO—O—(CH₂), —N(R₁₈R₁₉),
—CO—O—(CH₂)ᵤ—NH—CO—CH(NH₂)—R₂₀, or
—CO—O—(CH₂)_{w}—NH—CO—R₁₇,
R₅ is hydrogen, (C₁₋₄)alkyl, (C₃₋₄)cycloalkyl, amino, (C₁₋₄)alkylamino, di(C₁₋₄)alkylamino, aryl, e.g. (C₆₋₁₈)aryl, or heterocyclyl, e.g. having 5 or 6 ring members and 1 to 4 heteroatoms selected from N, O or S,
R₆ is hydrogen, (C₁₋₄)alkyl, (C₃₋₈)cycloalkyl, aryl, e.g. (C₆₋₁₈)aryl, (C₁₋₄)alkyl substituted by heterocyclyl having 5 or 6 ring members and 1 to 4 heteroatoms selected from N, O or S, amino(C₁₋₆)alkyl, (C₁₋₄)alkylamino(C₁₋₆)alkyl, di(C₁₋₄)alkylamino(C₁₋₆)alkyl, hydroxy(C₁₋₆)alkyl, hydroxy(C₁₋₄)alkylamino(C₁₋₆)alkyl or an amino acid residue, e.g. an amino acid residue of formula —CH₂—CH(NH₂)—COOH, or, e.g. an amino acid residue which amino acid residue is obtainable by reacting a compound of formula I wherein R₆ is —CO—(CH₂)ᵥ—CO—Cl with an amino acid, an amino acid mono(C₁₋₆)alkyl ester or an amino acid di(C₁₋₆)alkyl ester,
R₇ and R₈ independently of each other are (C₁₋₄)alkyl, (C₃₋₈)cycloalkyl, aryl or heterocyclyl having 5 or 6 ring members and 1 to 4 heteroatoms selected from N, O or S,
R₉ and R₁₀ independently of each other are hydrogen or (C₁₋₄)alkyl or
one of R₉ and R₁₀ is hydrogen and the other is (C₃₋₈)cycloalkyl, (C₁₋₄)alkyl, aryl, e.g. (C₆₋₁₈)aryl, or heterocyclyl having 5 or 6 ring members and 1 to 4 heteroatoms selected from N, O or S,
R₁₁ is (C₁₋₄)alkyl, —OR₁₂, —NR₁₃R₁₄, an amino acid residue, e.g. which amino acid residue is obtainable by reacting a compound of formula I wherein R₁₁ is —CO—(CHR₁₅)—O—(CH₂)ₒ—CO—Cl with an amino acid, an amino acid mono(C₁₋₆)alkyl ester or an amino acid di(C₁₋₆)alkyl ester,
R₁₂ is hydrogen or (C₁₋₄)alkyl, such as an amino acid residue, wherein the binding is effected via its amine group;
R₁₃ and R₁₄ independently of each other are hydrogen, (C₁₋₄)alkyl, amino(C₁₋₆)alkyl, (C₁₋₄)alkylamino(C₁₋₆)alkyl, di(C₁₋₄)alkylamino(C₁₋₆)alkyl,
R₁₅ is hydrogen or (C₁₋₄)alkyl,
R₁₆ is hydrogen, (C₁₋₄)alkyl, carboxyl or a carboxylic ester residue, e.g. attached via its carbonyl group;
R₁₇ is amino(C₁₋₄)alkyl, (C₁₋₄)alkylamino(C₁₋₄)alkyl or di(C₁₋₄)alkylamino(C₁₋₄)alkyl,
R₁₈ is hydrogen or (C₁₋₄)alkyl,
R₁₉ is hydroxy(C₁₋₄)alkyl,
R₂₀ is (C₁₋₄)alkyl or hydroxy(C₁₋₄)alkyl,
m is 0 to 4, n is 2 to 8, o is 0 to 4, p is 0 to 4, q is 1 to 8, r is 0 to 4, s is 1 to 4, t is 1 to 4, u is 1 to 6, and w is 1 to 6,
in free form or in the form of a salt.

13. The method of claim 12 wherein the phenyl-(4-phenyl-pyrimidin-2-yl)-amine is a compound of formula I, wherein R₁ is chloro, R₂ is hydrogen, R₃ is trifluoromethyl and R₄ is a group of formula —CO—O—(CH₂)₂—N[(C₂H₅OH)(CH₃)], in free form or in the form of a salt.

14. The method of claim 12 wherein the phenyl-(4-phenyl-pyrimidin-2-yl)-amine is a compound of formula I, wherein R₁ is chloro, R₂ is hydrogen, R₃ is trifluoromethyl and R₄ is hydrogen, in free form or in the form of a salt.

15. The pharmaceutical composition of claim 4, wherein the phenyl-(4-phenyl-pyrimidin-2-yl)-amine is a compound of formula 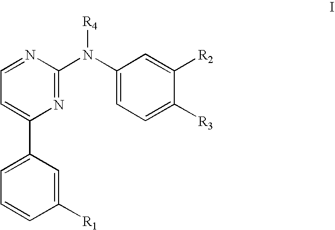
wherein
R₁ is halogen or halo(C₁₋₄)alkyl,
R₂ is hydrogen, halogen or halo(C₁₋₄)alkyl,
R₃ is halogen or halo(C₁₋₄)alkyl,
R₄ is hydrogen, (C₁₋₈)alkyl, hydroxy(C₁₋₄)alkyl or a group of formula
—CO—R₅,
—CO—(CH₂)ₘ—OR₆,
CO—CO—R₇,
—CO—CO—OR₈,
—CO—N(R₉R₁₀),
—CO—(CH₂)ₙ—CO—R₁₁,
—CO—(CHR₁₅)—O—(CH₂)ₒ—CO—R₁₁,
—CO—(CH₂)ₚ—O—(CH₂)_{q}—O—(CH₂)ᵣ—R₁₆,
—CO—O—(CH₂), —O—CO—R₁₇,
—CO—O—(CH₂), —N(R₁₈R₁₉),
—CO—O—(CH₂)ᵤ—NH—CO—CH(NH₂)—R₂₀, or
—CO—O—(CH₂)_{w}—NH—CO—R₁₇,
R₅ is hydrogen, (C₁₋₈)alkyl, (C₃₋₈)cycloalkyl, amino, (C₁₋₄)alkylamino, di(C₁₋₄)alkylamino, aryl, e.g. (C₆₋₁₈)aryl, or heterocyclyl, e.g. having 5 or 6 ring members and 1 to 4 heteroatoms selected from N, O or S,
R₆ is hydrogen, (C₁₋₄)alkyl, (C₃₋₈)cycloalkyl, aryl, e.g. (C₆₋₁₈)aryl, (C₁₋₄)alkyl substituted by heterocyclyl having 5 or 6 ring members and 1 to 4 heteroatoms selected from N, O or S, amino(C₁₋₄)alkyl, (C₁₋₄)alkylamino(C₁₋₆)alkyl, di(C₁₋₄)alkylamino(C₁₋₆)alkyl, hydroxy(C₁₋₆)alkyl, hydroxy(C₁₋₄)alkylamino(C₁₋₆)alkyl or an amino acid residue, e.g. an amino acid residue of formula —CH₂—CH(NH₂)—COOH, or, e.g. an amino acid residue which amino acid residue is obtainable by reacting a compound of formula I wherein R₆ is —CO—(CH₂), —CO—Cl with an amino acid, an amino acid mono(C₁₋₆)alkyl ester or an amino acid di(C₁₋)alkyl ester,
R₇ and R₈ independently of each other are (C₁₋₄)alkyl, (C₃₋₈)cycloalkyl, aryl or heterocyclyl having 5 or 6 ring members and 1 to 4 heteroatoms selected from N, O or S,
R₉ and R₁₀ independently of each other are hydrogen or (C₁₋₄)alkyl or one of R₉ and R₁₀ is hydrogen and the other is (C₃₋₈)cycloalkyl, (C₁₋₄)alkyl, aryl, e.g. (C₆₋₁₈)aryl, or heterocyclyl having 5 or 6 ring members and 1 to 4 heteroatoms selected from N, O or S,
R₁₁ is (C₁₋₄)alkyl, —OR₁₂, —NR₁₃R₁₄, an amino acid residue, e.g. which amino acid residue is obtainable by reacting a compound of formula I wherein R₁₁ is —CO—(CHR₁₅)—O—(CH₂)ₒ—CO—Cl with an amino acid, an amino acid mono(C₁₋₆)alkyl ester or an amino acid di(C₁₋₆)alkyl ester,
R₁₂ is hydrogen or (C₁₋₄)alkyl, such as an amino acid residue, wherein the binding is effected via its amine group;
R₁₃ and R₁₄ independently of each other are hydrogen, (C₁₋₄)alkyl, amino(C₁₋₆)alkyl, (C₁₋₄)alkylamino(C₁₋₆)alkyl, di(C₁₋₄)alkylamino(C₁₋₆)alkyl,
R₁₅ is hydrogen or (C₁₋₄)alkyl,
R₁₆ is hydrogen, (C₁₋₄)alkyl, carboxyl or a carboxylic ester residue, e.g. attached via its carbonyl group;
R₁₇ is amino(C₁₋₄)alkyl, (C₁₋₄)alkylamino(C₁₋₄)alkyl or di(C₁₋₄)alkylamino(C₁₋₄)alkyl,
R₁₈ is hydrogen or (C₁₋₄)alkyl,
R₁₉ is hydroxy(C₁₋₄)alkyl,
R₂₀ is (C₁₋₄)alkyl or hydroxy(C₁₋₄)alkyl,
m is 0 to 4, n is 2 to 8, o is 0 to 4, p is 0 to 4, q is 1 to 8, r is 0 to 4, s is 1 to 4, t is 1 to 4, u is 1 to 6, and w is 1 to 6,
in free form or in the form of a salt.

16. The pharmaceutical composition of claim 15, wherein the phenyl-(4-phenyl-pyrimidin-2-yl)-amine is a compound of formula I, wherein R₁ is chloro, R₂ is hydrogen, R₃ is trifluoromethyl and R₄ is a group of formula —CO—O—(CH₂)₂—N[(C₂H₅OH)(CH₃)], in free form or in the form of a salt.

17. The pharmaceutical composition of claim 15, wherein the phenyl-(4-phenyl-pyrimidin-2-yl)-amine is a compound of formula I, wherein R₁ is chloro, R₂ is hydrogen, R₃ is trifluoromethyl and R₄ is hydrogen, in free form or in the form of a salt.

18. The process according to claim 5, wherein the phenyl-(4-phenyl-pyrimidin-2-yl)-amine is a compound of formula 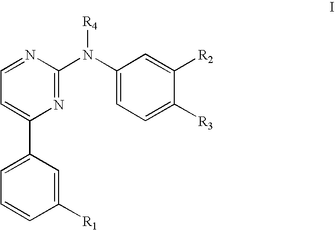
wherein
R₁ is halogen or halo(C₁₋₄)alkyl,
R₂ is hydrogen, halogen or halo(C₁₋₄)alkyl,
R₃ is halogen or halo(C₁₋₄)alkyl,
R₄ is hydrogen, (C₁₋₄)alkyl, hydroxy(C₁₋₆)alkyl or a group of formula
—CO—R₅,
—CO—(CH₂)ₘ—OR₆,
—CO—CO—R₇,
—CO—CO—OR₈,
—CO—N(R₉R₁₀),
—CO—(CH₂)ₙ—CO—R₁₁,
—CO—(CHR₁₅)—O—(CH₂)ₒ—CO—R₁₁,
—CO—(CH₂)ₚ—O—(CH₂)_{q}—O—(CH₂)ᵣ—R₁₆,
—CO—O—(CH₂)ₛ—O—CO—R₁₇,
—CO—O—(CH₂)ₜ—N(R₁₈R₁₉),
—CO—O—(CH₂)ᵤ—NH—CO—CH(NH₂)—R₂₀, or
—CO—O—(CH₂)_{w}—NH—CO—R₁₇,
R₅ is hydrogen, (C₁₋₈)alkyl, (C₃₋₄)cycloalkyl, amino, (C₁₋₄)alkylamino, di(C₁₋₄)alkylamino, aryl, e.g. (C₆₋₁₈)aryl or heterocyclyl, e.g. having 5 or 6 ring members and 1 to 4 heteroatoms selected from N, O or S,
R₆ is hydrogen, (C₁₋₄)alkyl, (C₃₋₈)cycloalkyl, aryl, e.g. (C₁₋₁₈)aryl, (C₁₋₄)alkyl substituted by heterocyclyl having 5 or 6 ring members and 1 to 4 heteroatoms selected from N, O or S, amino(C₁₋₆)alkyl, (C₁₋₄)alkylamino(C₁₋₁₆)alkyl, di(C₁₋₄)alkylamino(C₁₋₆)alkyl, hydroxy(C₁₋₆)alkyl, hydroxy(C₁₋₄)alkylamino(C₁₋₆)alkyl or an amino acid residue, e.g. an amino acid residue of formula —CH₂—CH(NH₂)—COOH, or, e.g. an amino acid residue which amino acid residue is obtainable by reacting a compound of formula I wherein R₆ is —CO—(CH₂), —CO—Cl with an amino acid, an amino acid mono(C₁₋₄)alkyl ester or an amino acid di(C₁₋₆)alkyl ester,
R₇ and R₈ independently of each other are (C₁₋₄)alkyl, (C₃₋₈)cycloalkyl, aryl or heterocyclyl having 5 or 6 ring members and 1 to 4 heteroatoms selected from N, O or S,
R₉ and R₁₀ independently of each other are hydrogen or (C₁₋₄)alkyl or
one of R₉ and R₁₀ is hydrogen and the other is (C₃₋₈)cycloalkyl, (C₁₋₄)alkyl, aryl, e.g. (C₆₋₁₈)aryl, or heterocyclyl having 5 or 6 ring members and 1 to 4 heteroatoms selected from N, O or S,
R₁₁ is (C₁₋₄)alkyl, —OR₁₂, —NR₁₃R₁₄, an amino acid residue, e.g. which amino acid residue is obtainable by reacting a compound of formula I wherein R₁₁ is —CO—(CHR₁₅)—O—(CH₂)ₒ—CO—Cl with an amino acid, an amino acid mono(C₁₋₆)alkyl ester or an amino acid di(C₁₋₄)alkyl ester,
R₁₂ is hydrogen or (C₁₋₄)alkyl, such as an amino acid residue, wherein the binding is effected via its amine group;
R₁₃ and R₁₄ independently of each other are hydrogen, (C₁₋₄)alkyl, amino(C₁₋₆)alkyl, (C₁₋₄)alkylamino(C₁₋₆)alkyl, di(C₁₋₄)alkylamino(C₁₋₆)alkyl,
R₁₅ is hydrogen or (C₁₋₄)alkyl,
R₁₆ is hydrogen, (C₁₋₄)alkyl, carboxyl or a carboxylic ester residue, e.g. attached via its carbonyl group;
R₁₇ is amino(C₁₋₄)alkyl, (C₁₋₄)alkylamino(C₁₋₄)alkyl or di(C₁₋₄)alkylamino(C₁₋₄)alkyl,
R₁₈ is hydrogen or (C₁₋₄)alkyl,
R₁₉ is hydroxy(C₁₋₄)alkyl,
R₂₀ is (C₁₋₄)alkyl or hydroxy(C₁₋₄)alkyl,
m is 0 to 4, n is 2 to 8, o is 0 to 4, p is 0 to 4, q is 1 to 8, r is 0 to 4, s is 1 to 4, t is 1 to 4, u is 1 to 6, and w is 1 to 6,
in free form or in the form of a salt.

19. The process according to claim 18, wherein the phenyl-(4-phenyl-pyrimidin-2-yl)-amine is a compound of formula I, wherein R₁ is chloro, R₂ is hydrogen, R₃ is trifluoromethyl and R₄ is a group of formula —CO—O—(CH₂)₂—N[(C₂H₅OH)(CH₃)], in free form or in the form of a salt.

20. The process according to claim 18, wherein the phenyl(4-phenyl-pyrimidin-2-yl)-amine is a compound of formula I, wherein R₁ is chloro, R₂ is hydrogen, R₃ is trifluoromethyl and R₄ is hydrogen, in free form or in the form of a salt.
